SEQUENCE LISTING (22 sequences)

SEQ ID NO: 1 - DNA, 1437 bp, Homo sapiens
  misc_feature: Human melanopsin
atgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctcagctggatctccatacggaggcgccaggagtccctgggctcggagagtgaggtgggctggacacacatggaggcagcagctgtgtggggagctgcccagcaagcaaatgggcggtccctctacggtcagggtctggaggacttggaagccaaggcaccccccagaccccagggacacgaagcagagactccagggaagaccaaggggctgatccccagccaggaccccaggatgtag

SEQ ID NO: 2 - protein, 308 aa, Homo sapiens
  MISC_FEATURE: Human melanopsin
MNSGRVSTSCMATASWWDSSSSSSGRSSTAGTWAAAWVTVDVDHAHYTGTVVGTGMGNTVYTCRSRSRTANMNAVSDMSTAVTSSYKWGTGCYACGAGSSMTTAADRYVTRATGVASKRRAAVGVWYAAWSGWSAYVGTSCSWDYMSTAVRAYTMCCVYCYRARTGRATGACKGNGSWRRSCKMAKMVVSWAYSAVAVAAGYAHVTYMSSVAVAKASAHNYATHKYRVAAHCGVGVSRRHSRYSYRSTHRSTTSHTSNSWSRRRSGSSVGWTHMAAAVWGAAANGRSYGGDAKARGHATGKTKGSDRM

SEQ ID NO: 3 - DNA, 29 bp, Artificial Sequence
  Synthetic Primer
ccggaattcatcccaactcaggatgaacc

SEQ ID NO: 4 - DNA, 28 bp, Artificial Sequence
  Synthetic Primer
tgctctagacgtcctacatcctggggtc

SEQ ID NO: 5 - DNA, 32 bp, Artificial Sequence
  Synthetic Primer
gaagatctcatcccaactcaggatgaaccctc

SEQ ID NO: 6 - DNA, 56 bp, Artificial Sequence
  Synthetic Primer
cggaattcctagtgatggtgatggtgatgcatcctggggtcctggctggggatcag

SEQ ID NO: 7 - DNA, 20 bp, Artificial Sequence
  Synthetic Primer
tcaagccggaggtcaacaac

SEQ ID NO: 8 - DNA, 20 bp, Artificial Sequence
  Synthetic Primer
tcttggacacggtagcagag

SEQ ID NO: 9 - DNA, 18 bp, Artificial Sequence
  Synthetic Primer
cagccttcatgggatttg

SEQ ID NO: 10 - DNA, 18 bp, Artificial Sequence
  Synthetic Primer
gtgcatgcttggagttga

SEQ ID NO: 11 - DNA, 20 bp, Artificial Sequence
  Synthetic Primer
caagcatcttcacctatacc

SEQ ID NO: 12 - DNA, 18 bp, Artificial Sequence
  Synthetic Primer
cgctgaacacgtctgggc

SEQ ID NO: 13 - DNA, 19 bp, Artificial Sequence
  Synthetic Primer
atcctgctcctgggactac

SEQ ID NO: 14 - DNA, 19 bp, Artificial Sequence
  Synthetic Primer
tcttggccatcttgcactc

SEQ ID NO: 15 - DNA, 24 bp, Artificial Sequence
  Synthetic Primer
cctgatgtaggacgaagaatgacc

SEQ ID NO: 16 - DNA, 23 bp, Artificial Sequence
  Synthetic Primer
cacaagcacacgatggaataagg

SEQ ID NO: 17 - DNA, 24 bp, Artificial Sequence
  Synthetic Primer
gaggggtgacagaaacttcatcag

SEQ ID NO: 18 - DNA, 22 bp, Artificial Sequence
  Synthetic Primer
ttgtggagacagacactgccag

SEQ ID NO: 19 - DNA, 19 bp, Artificial Sequence
  Synthetic Primer
gagaagcactccagagagc

SEQ ID NO: 20 - DNA, 19 bp, Artificial Sequence
  Synthetic Primer
ttgagctggtattctgagg

SEQ ID NO: 21 - DNA, 19 bp, Artificial Sequence
  Synthetic Primer
agtggcatcagtgctgagg

SEQ ID NO: 22 - DNA, 19 bp, Artificial Sequence
  Synthetic Primer
cgagtcattgagctggaac

(19) United States
(12) Patent Application Publication
(10) Pub. No.: US 2009/0208462 A1
(43) Pub. Date: Aug. 20, 2009

(54) Methods of Treatment

(75) Inventors: Mark W. Hankins, London (GB); Robert Lucas, Manchester (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: IMPERIAL INNOVATIONS LIMITED, London (GB)

(21) Appl. No.: 11/791,816

(22) PCT Filed: Nov. 29, 2005
(86) PCT No.: PCT/GB05/04559

§ 371 Date: Feb. 1, 2008

(30) Foreign Application Priority Data
Nov. 30, 2004 (GB) 0426196.2

(51) Int. Cl. A61K 35/12; C12N 5/06; C12N 15/87; C12N 15/63; C12N 5/10; A61K 31/7088; A61P 27/02

(52) U.S. Cl. 424/93.7; 435/375; 435/455; 435/320.1; 435/325; 514/44 R

(57) ABSTRACT

The invention concerns the use of a nucleic acid encoding a photoactivator in the manufacture of a medicament for inducing photosensitivity in neuronal cells. In particular, the invention concerns the induction of photosensitivity in retinal ganglion cells (RGCs) for restoring sight and/or alleviating blindness in an individual. The invention further concerns vectors, cells and pharmaceutical compositions therefor.

The present invention provides the use of a photoactivator for inducing photosensitivity in one or more neuronal cell; wherein the photoactivator is capable of activating a photo-transduction cascade in a neuronal cell in response to light.

In particular, the present invention relates to agents and methods for restoring sight and/or alleviating blindness in an individual, particularly by using a photoactivator to induce photosensitivity in one or more retinal ganglion cell (RGC).

The eyes are complex sense organs comprising a layer of receptors, a lens system that focuses light on these receptors and a system of nerves that conducts impulses from the receptors to the brain. The visual receptors (known as rods and cones) and four types of neurons (bipolar cells, ganglion cells. horizontal cells and amacrine cells) are contained in the retina at the back of the eye. The rods and cones synapse with bipolar cells, which in turn synapse with ganglion cells, the axons of which converge and leave the eye as the optic nerve.

Light is absorbed by photosensitive substances in the rods and cones. Light absorption induces a conformational change in the structure of these substances and triggers a sequence of events that transmits a signal to the brain. The photosensitive substances in the rods and cones of humans and most other mammals are made up of a protein called an opsin, and retinal₁, the aldehyde of vitamin A₁ (Filipek et al. 2003; Yokoyama, 2000). Opsin-like molecules have also been described in fish and other vertebrates (Koyanagi et al., 2004) The photosensitive substance in rods is called rhodopsin that comprises an opsin called rodopsin, which is a G protein-coupled seven-transmembrane receptor, to which retinal₁ is attached. Cones possess a distinct photosensitive substance that is similar in structure to rhodopsin.

Upon exposure to light, the retinal in rhodopsin is converted from an 11-cis configuration to an all-trans isomer. This induces a confrontational change in the structure of the opsin that activates a heterotrimeric G protein called transducin or Gₜ₁ which is associated with the intracellular domains of the classical rod and cone opsins. The G protein exchanges GDP for CTP, allowing the α-subunit to dissociate from the βγ-subunits and activate downstream effectors, such as cGMP phosphodiesterase, which result in the generation of a signal. All-trans retinal, is subsequently released from activated rhodopsin and the opsin associates with 11-cis retinal₁ that is produced by cells of the retinal pigment epithelium (RPE) to regenerate inactive rhodopsin. The cellular components and events involved in the generation, transmission and amplification of an intracellular signal following activation of the opsin are collectively termed the photo-transduction cascade (Stryer, 1991).

One of the most surprising recent findings in the vertebrate retina is the emergence of a parallel light-sensing pathway that is able to function independently of the classical rod and cone photoreceptors. It is now well established that an extremely small proportion (less than 5% of the total) of retinal ganglion cells (RGCs) are themselves directly light-sensitive (Lucas et al., 1999; Berson et al., 2001; Provencio et al., 1998). This distinct subset of cells are known as intrinsically photosensitive retinal ganglion cells (ipRGCs) and are capable of translating light information into a biological signal in the complete absence of the classical rod and cone cells (Lucas et al., 1999; Lucas et al., 2001; Freedman et al., 1999; Czeisler et al., 1995).

Light detection in ipRGCs appears to involve an opsin photosensitive substance called melanopsin that is quite different from rod and cone opsins (Provensio et al., 2000; Hannibal et al., 2002) and that is structurally more similar to opsins found in invertebrates. Importantly7, invertebrate opsins drive depolarising responses (Hardie, 1991) and also have an endogenous photoisomerase activity that allows them to reconstitute their chromophore in situ (Hillman et al., 1983). Consequently, melanopsin-expressing cells depolarise in response to light and function at a distance from the RPE (Berson et al., 2001; Hannibal et al., 2002; Gooley et al., 2001).

The function of ipRGCs appears to be the regulation of time-of-day dependent photoresponses such as circadian entrainment and pupillary constriction (Lucas et al., 1999; Lucas et al., 2001; Czeisler et al., 1995; Brainard et al., 2001). Thus, their projections extend to those brain areas that adapt behaviour and physiology according to time of day but not those generating visual images (Provencio et al., 1998; Hannibal et al., 1997). This separation is reflected in reports that some blind subjects with no perception of light retain normal day:night responses (Czeisler et al., 1995; Lockley et al., 1997).

Hereditary degenerative diseases affecting rod and cone photoreceptors are the second largest cause of blindness in the developed world (Inglehearn, 1998). Whilst these conditions may be characterised by a catastrophic loss of light-sensitive rod and cone cells in the outer retina of the eye, sufferers generally retain a normal optic apparatus and a viable population of the retinal ganglion cells that form the optic nerve with intact projections to the higher visual areas.

These features raise the possibility that clinical interventions aimed at overcoming the primary lesion (i.e. the loss of photoreceptive capacity) will have a significant impact on visual function. Consequently, a great deal of research has gone into developing interventions that either slow photoreceptor degeneration (for example, by gene therapy or retinal transplants) or restore photosensitivity to an already degenerate tissue (for example, by implantation of electronic prostheses in direct electrical contact with the surviving retinal ganglion cells). All have met with some degree of success in animal studies (Bennett, 2000; Lund et al., 2001; Zrenner et al., 1999; Chow et al., 2001).

The approach that has the potential for widest applicability is that of retinal prosthetics. Here, a physical substrate replaces the absent photoreceptors by translating light into an electrical signal and passing this directly to local RGCs (Zrenner et al., 1999; Zrenner et al., 1997; Schlosshauer et al., 1999). There are three substantial problems that this technology has yet to fully address. The first is that of surgical access and biocompatibility. The second is the long-term efficiency of information transfer from the physical prosthesis to the RGCs. The third is a lack of spatial resolution associated with the relatively low density of electrodes available using current technology.

Accordingly, new agents and methods capable of restoring sight and/or alleviating blindness are highly desired.

In one aspect, the present invention provides the use of a photoactivator for inducing photosensitivity in one or more retinal ganglion cell, wherein the photoactivator is capable of activating a photo-transduction cascade in a retinal ganglion cell in response to light.

The present invention thus provides a new approach to induce photosensitivity in the dystrophic retina by inducing photosensitivity in retinal ganglion cells. Because this approach transforms the RGCs into cells capable of both translating light exposure into a biological signal, and conveying that signal to the visual centres of the brain, it provides a simple approach to the treatment of blindness.

It will be understood that this aspect of the invention includes the use of a photoactivator for inducing photosensitivity in one or more neuronal cell (e.g. RGC) in vivo (for example, in an individual) and/or ex vivo (for example, outside the body of an individual) and/or in vitro (for example, in a cell culture).

It is not possible to employ key elements of rod/cone photo-transduction cascades because the nature of these cascades males them ill-suited to the task. In these cells, light absorption is carried out by a photosensitive substance comprising an 11-cis reinaldehyde chromophore bound by an opsin protein. Light induces an isomerisation of the chromophore to all-trans retinaldehyde resulting in activation of a well-defined G protein-coupled transduction cascade. This culminates in the closure of cation channels on the cell surface and a consequent hyperpolarisation of the photoreceptor. Thee aspects of the cascade render it unsuitable for the task of inducing photosensitivity in RGCs. The first is that rod and cone cells rely on a steady supply of 11-cis retinaldehyde from the RPE to replace the chromophore bleached by light exposure, and RGCs are too distant from the RPE to rely on this source. Secondly, the light dependent hyperpolarisation of rod and cone cells is incompatible with generation of the action potentials by which RGCs transmit information to the brain. A third problem (particularly for cone opsins) is that they appear to rely on specific factors within the photoreceptor cell to reliably attain a functional conformation Heterologous expression in other cells types is rarely consistent with the generation of photosensitive pigment.

It will be appreciated that the invention also provides a use for inducing photosensitivity in other neuronal cells. In doing so, the invention provides a means of inducing photosensitivity in neuronal cells that usually have no or low photosensitivity. Neuronal cells that have been rendered photosensitive using the present invention may be physiologically activated (typically characterised by changes in membrane potential) using light (photic) stimulation.

In the first aspect, the present invention provides the use of a photoactivator for inducing photosensitivity in one or more neuronal cell, wherein the photoactivator is capable of activating a photo-transduction cascade in a neuronal cell in response to light.

Preferably, the invention provides a use wherein the neuronal cell is a retinal ganglion cell (RGC).

Primary and transformed cell cultures can be prepared from neural tissue according to methods known in the art, and many are commercially available. For example, mouse neuroblastoma-2a (Neuro-2a) cell line can be obtained from the ATCC (American Type Culture Collection; European distributors http://www.Igcpromochem.com/atcc, ATCC number CCL-131).

It will be understood that types of neuronal cell other than a retinal ganglion cell (RGC) could be used in the method of the invention. By neuronal cell we include cells of the nervous system of a mammal, particularly cells of the central nervous system (CNS) which lie within the blood brain barrier and/or the blood-retina barrier, especially cells of the brain (e.g. neurons from spinal cord, cerebellum, basal ganglia, thalamus, hippocampus, substantia nigra, neocortex, endothelial cells derived from the neural crest, foetal neurons, neuronal multipotent cell lines, adrenal chromaffin cells, striatum, glial cells, myoblasts, or fibroblasts).

The specific application of the invention will depend upon the cell type targeted and the host employed.

The invention has therapeutic potential for reversing sensory deficits. For example, inducing photosensitivity in ON bipolar cells in the retina may contribute to the alleviation of blindness and/or the restoration of sight. ON bipolar cells are those cells that depolarise in response to light, which differentiates them from OFF bipolar cells that hyperpolarise in response to light. ON bipolar cells also drive the ON-centre ganglion cells that depolarise and generate action potentials in response to light at their receptive field centre. Being able to selectively target this Croup of ganglion cells could improve retinal stimulation.

Inducing photosensitivity in cells of the auditory system may provide an opportunity for alleviating deafness and/or restoring hearing. In this case, a suitable optical interface would translate sound into a light signal which would be used to stimulate photosensitised neurones in the cochlea of the ear.

Long-term depolarisation induces apoptosis and the ability to induce photosensitivity in specific cell types raises the possibility of using light to selectively ablate individual (e.g. cancerous) cells in an otherwise healthy tissue. In an experimental setting, this approach will allow investigations of the effects of either selective ablation (long-term light exposure) or excitation of specific cell types. Cancerous cells can express a variety of ion channels (Huang et al., 2004) which could effect long-term depolarisation (and therefore apoptosis) of photosensitive cancerous cells in response to light stimulation. In addition, light activation of photosensitive cells can induce mobilisation of intracellular calcium ions which could lead to long-term depolarisation and apoptosis without the need for ion channels.

A number of techniques known to those in the art could be used to induce activity of a photoactivator in selected populations of specific cell types. For example, a photoactivator, or a nucleic acid encoding a photoactivator, could be introduced into specific tissues and/or cell types in vivo and/or in vivo by local inoculation with a viral and/or plasmid expression construct.

Alternatively, a gene-specific promoter could be used to direct expression of a nucleic acid encoding a photoactivator in cells in which that promoter is active. For example, expression could be restricted to retinal ON bipolar cells by using the cell-specific mGluR6 promoter. DNA comprising a cell-specific promoter and a nucleic acid encoding a photoactivator could be introduced into cells in plasmid and/or viral vectors using techniques known in the art.

Alternatively, transgenic animals could be generated in which a photoactivator was expressed in specific cell types in that animal. Techniques for the generation of a number of species of transgenic animal (including, for example, zebrafish, drosophila and mouse and rat species) are well known to those skilled in the art. Nucleic acid constructs that are suitable for the introduction of a nucleic acid of interest are known and can be randomly incorporated into the genome of cells of a transgenic animal or introduced at specific loci into the genome of cells of a trans genic animal by homologous recombination (‘knock-ins’).

A nucleic acid comprising a promoter and the nucleic acid of interest may be introduced into the genome of cells of the transgenic animal flanked by enzyme recognition sites that allow the nucleic acid to be selectively spliced out of the genome of cells of the transgenic animal. Such systems are well known in the art and allow a nucleic acid of interest to be turned “on” and “off” in selected cell types in the transgenic animal. A commonly used system is the “lox” system, wherein the nucleic acid of interest is flanked by recognition sites (termed “lox” sites) that can be recognised by the cre recombinase enzyme.

By “photoactivator” we include a molecule or complex of molecules that is activated in response to light exposure and is capable of simulating cellular components of a photo-transduction cascade to generate an intracellular signal and/or a signal indicative of light exposure that can be transmitted to neighboring cells and/or the brain of an individual. As defined in the present invention, a photoactivator may be, for example, a single molecule (such as a protein) or a molecule complexed with other molecules (such as a chromophore). Examples of chromophores include 11-cis retinal, 9-cis retinal and all-trans retinal.

Photoactivators of use in the present invention include members of the opsin gene family such as, for example, melanop sin, vaopsin, pinopsin, parapinopsin, rod opsin, cone opsins, TMT opsin, neuropsin (OPN5), and opsins from photoreceptive structures of invertebrate species.

By “photo-transduction cascade” we include the cellular events and components involved in the generation, amplification, transmission and termination of an intracellular signal following activation of an opsin in response to light stimulation.

By “activating a photo-transduction cascade” we include the activation of one or more cellular components of the photo-transduction cascade which subsequently leads to the generation, amplification and transmission of an intracellular signal by the components of the photo-transduction cascade.

The term “retinal ganglion cells” is a generic term which includes all of the output neurons of the retina, the vast majority of which (for example 99%) project to the visual areas of the brain. In classical physiology these include ‘ON’ and ‘OFF’ cells that are respectively excited or inhibited by light presented at the receptive filed centre.

By “in response to light” we include a response to light stimuli in the cone (photopic) brightness range and to the presentation of electromagnetic radiation of wavelength within the range 300 to 900 nm.

By “inducing photosensitivity” we include inducing the ability of a cell (or cells) that is not sensitive to light or that has a relatively low sensitivity to light, to detect and/or respond to light.

For example, photosensitivity may be induced by introducing and expressing gene(s) encoding component(s) involved in a photo-transduction cascade into a cell, or by inducing the expression of gene(s) encoding component(s) involved in a photo-transduction cascade which are usually present in the genome of a cell but which are not usually expressed (for example, due to transcriptional or translational silencing of the gene).

Measuring and/or determining an increase in photosensitivity can include measuring the depolarisation of transformed cells in culture and/or by examining the behavioural and/or physiological responses of a whole organism in which the cell (or cells) of interest are present. Such methods are well known to those skilled in the arts of, for example, molecular biology, neurobiology and/or zoology. Cellular depolarisation may be measured electro-physiologically using single electrodes or by imaging cells or tissue slices in culture, using a combination of potentiometric and calcium dyes. In the case of chronic stimulation assays measuring and/or determining c-FOS expression or cell death may be used.

Preferably; the present invention provides a use wherein the photoactivator is a photoactivator which is present in an intrinsically photosensitive retinal ganglion cell (ipRGC).

Intrinsically photosensitive retinal ganglion cells (ipRGCs) comprise a morphologically homogenous class of retinal ganglion cells that respond to light in isolation from other neurones. They are involved in the regulation of non-image forming visual responses through projections to such brain areas as the suprachiasmatic nuclei and the pretectum.

Preferably, the present invention provides a use wherein the photoactivator is an opsin.

Opsins are light-activated G protein-coupled receptors that are found in photosensitive cells of vertebrates and invertebrates. Opsins are typically seven transmembrane receptors and are typically associated with a chromophore to form a photosensitive substance.

By “opsins” we include all G protein-coupled receptors that share at least 20% deduced amino acid similarity with bovine rod opsin. Members of the opsin family include vaopsin, pinopsin, parapinopsin, rod opsin, cone opsins, TMT opsin, neuropsin (OPN5), and opsins from photoreceptive structures of invertebrate species and non-mammalian vertebrate species (such as lamprey parapinopsin).

We also include artificial or modified forms of opsins which have, for example, different spectral sensitivity, altered sensitivity to light or altered activation/deactivation kinetics.

Preferably, the present invention provides a use wherein the photoactivator is an opsin and one or more co-factor of the opsin.

By “co-factor of the opsin” we include co-factors that opsins require in order to function, such as a chromophore.

Preferably, the present invention provides a use wherein the photoactivator is a mammalian opsin.

By mammalian opsin we include opsins encoded by the mammalian genome.

Preferably, the present invention provides a use wherein the photoactivator is a human opsin.

Mammalian rod and cone opsins are found exclusively in rod and cone photoreceptor cells of the retina and are responsible for their photosensitivity. Members of the opsin family found in the mammalian genome include: RGR opsin (which is expressed in the retinal pigment epithelium and retinal Muller cells and is involved in recycling bleached chromophore); per opsin (which is expressed in the retinal pigment epithelium and is involved in recycling bleached chromophore); melanopsin (which is expressed in intrinsically photosensitive retinal ganglion cells and performs an as yet unknown but essential role in their photosensitivity); encephalopsin (which is expressed in many tissues and performs an as yet unknown function); neuropsin/OPN5 (which is expressed in the eye and central nervous system and performs an as yet unknown function).

Preferably, the present invention provides a use wherein the photoactivator is melanopsin.

Melanopsin is a protein whose predicted amino acid sequence (based on cDNA sequence) shows significant similarity (>20% identity) with members of the opsin family. It is predicted to form a membrane bound heptahelical protein. It is expressed in a subset of retinal ganglion cells. The primary projection of these cells is to the suprachiasmatic nuclei. The precise role of melanopsin remains unknown, but retinal ganglion cells that contain this protein are intrinsically-photosensitive and disruption of the melanopsin gene abolishes this photosensitivity. The nucleotide sequence of the complete coding sequence of human melanopsin is shown in SEQ ID NO:1; the amino acid sequence of human melanopsin is shown in SEQ ID NO:2.

Preferably, the present invention provides a use wherein the photoactivator according to the earlier aspects of the invention is produced by expressing a nucleic acid encoding the photoactivator.

By “nucleic acid” we include single-stranded and/or double-stranded molecules of DNA (deoxyribonucleic acid) and/or RNA (ribonucleic acid) and derivatives thereof.

Preferably, the present invention provides a method wherein the nucleic, acid comprises:

(i) the nucleotide sequence of SEQ ID NO: 1; or

(ii) a nucleotide sequence which encodes an amino acid sequence with more than 50% identity to the deduced amino acid sequence of SEQ ID NO:1 or a nucleotide sequence which hybridises to the nucleotide sequence of SEQ ID NO:1 under stringent, or moderately stringent conditions; or

(iii) a nucleotide sequence which encodes an amino acid sequence with more than 20% identity to a sequence of at least 200 amino acids of bovine rod opsin; or

(iv) a fragment of the nucleotide sequence of SEQ ID NO:1 encoding a polypeptide fragment effective to induce photosensitivity in a retinal ganglion cell (RGC).

It is well known in the art that two nucleic acids encoding the same gene may have similar but non-identical nucleotide sequences. A variation in the nucleotide sequence of a gene is one which is (i) usable to produce a protein or a fragment thereof which is in turn usable to prepare antibodies which specifically bind to the protein encoded by the said gene or (ii) an antisense sequence corresponding to the gene or to a variation of type (i) as Just defined. For example, different codons can be substituted which code for the same amino acid(s) as the original codons. Alternatively, the substitute codons may code for a different amino acid that will not affect the activity or immunogenicity of the protein or which may improve its activity or immunogenicity. For example, site-directed mutagenesis or other techniques can be employed to create single or multiple mutations, such as replacements, insertions, deletions, and transpositions, as described in Botstein et al. (1985), which is incorporated herein by reference. Since such modified genes can be obtained by the application of known techniques to the teachings contained herein, such modified genes are within the scope of the claimed invention.

Moreover, it will be recognised by those skilled in the art that the gene sequence (or fragments thereof) of the invention can be used to obtain other DNA sequences that hybridise with it under conditions of high stringency. Such DNA includes any genomic DNA. Accordingly, the gene of the invention includes DNA that encodes an amino acid sequence with more than 50% identity to the deduced amino acid sequence of the gene identified in the method of the invention, or a DNA sequence that shows at least 55 per cent, preferably 60 per cent, and most preferably 70 per cent homology with the gene identified in the method of the invention, provided that such homologous DNA is usable in the methods of the present invention. The gene of the invention also includes DNA that encodes an amino acid sequence with more than 20% identity to a sequence of at least 200 amino acids of bovine rod opsin.

DNA-DNA, DNA-RNA and RNA-RNA hybridisation may be performed in aqueous solution containing between 0.1XSSC and 6XSSC and at temperatures of between 55° C. and 70° C. It is well known in the art that the higher the temperature or the lower the SSC concentration the more stringent the hybridisation conditions. By “high stringency” we mean 2XSSC and 65° C. 1XSSC is 0.15 M NaCl/0.015 M sodium citrate.

Variations of the gene include genes in which relatively short stretches (for example 20 to 50 nucleotides) have a high degree of homology (at least 50% and preferably at least 90 or 95%) with equivalent stretches of the gene of the invention even though the overall homology between the two genes may be much less. This is because important active or binding sites may be shared even when the general architecture of the protein is different.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperatures. The higher the degree of desired homology between the probe and hybridisable sequence, the higher the relative temperature that can be used. For additional details and explanation of stringency of hybridization reactions, see Ausubel et al. (1995) or Protocols Online URL: www.protocol-online.net/molbio/index.htm).

“Stringent conditions” or high-stringency may be identified by those that: (1) use low ionic strength and high temperature for washing, for example 0.1X SSC, 0.2% SDS @ 65-70° C.

“Moderately-stringent conditions” may be identified as described by Sambrook et al. (2001), and include the use of washing solution and hybridization conditions (e.g. temperature, ionic strength, and % SDS) less stringent that those described above. An example of moderately stringent conditions is 0.2X SSC, 0.1% SDS @ 58-65° C. The skilled artisan will recognize how to adjust temperature, ionic strength, etc. as necessary to accommodate factors such as probe length, degree of homology between probe and target site and the like. Therefore, in addition to the sequence of interest, it is contemplated that additional or alternative probe sequences which vary from that of the sequence of interest will also be useful in screening for the sequence of interest.

Homologues or orthologues of opsins such as melanopsin may be of use in this aspect of the invention. Orthologues melanopsin have been identified in mice (Genbank Accession number AF147789), rat (AY072689.1), zebrafish (AY078161), Xenopus laevis (AF014797), Chicken (AY036061) and roach (AY226847).

Nucleotide sequences encoding fragments of opsins such as melanopsin (or orthologues or homologues thereof or encoding mutated versions of opsins such as melanopsin (or orthologues or homologues thereol are also thought to be of use in the present invention. Particularly preferred mutant forms of opsins such as melanopsin include those with mutations in the C-terminal (intracellular domain) or N-terminal (extracellular domain).

Preferably, the present invention provides a use wherein the nucleic acid comprises the nucleic acid encoding a photoactivator in a vector.

By “vector” we include a vehicle used in gene cloning and/or gene expression to introduce a nucleic acid of interest into a host cell, bacteriophage, virus or yeast. The nucleic acid of interest may be joined to a aside variety Of vectors for introduction into an appropriate host. The vector will depend upon the nature of the host, the manner of the introduction of the vector into the host, and whether episomal maintenance or integration is desired.

For example, in bacterial hosts three different types of vector can be used: bacteriophage, cosmids, plasmids and their hybrid derivatives. In these vectors, the nucleic acid of interest can be spliced into the vector using specific restriction enzymes and ligases, or by using homologous recombination, although other methods will be well known by those in the art. In some phage vectors part of the viral genome may be removed and replaced with the nucleic acid of interest.

Preferred vectors for use in the invention include adeno-associated vector. This is a viral strain of choice for gene therapy because of its safety and efficacy and has been shown to target retinal ganglion cells upon intravitreal injection. A particularly preferred vector is the recombinant adeno-associated virus vector based on the rAVE expression cassette (available from Genedetect—www.genedetect.com).

Alternatively, nucleic acid of the invention may be delivered to a cell of interest without the use of a vector, for example by electroporation of the nucleic acid as described in Matsuda et al. (2004).

More preferably, the present invention provides a use wherein the vector is an expression vector.

By “expression vector” we include vectors that possess regions of nucleotide sequence that direct transcription and/or translation of the nucleic acid of interest such that the protein(s) encoded by the nucleic acid is expressed. If necessary, the nucleic acid of the invention may be linked to the appropriate transcriptional and translational regulatory control nucleotide sequences recognised by the desired host, although such controls are generally available in expression vectors. Thus, the nucleic acid of the invention may be operatively linked to an appropriate promoter. Bacterial promoters include the E. coli lacI and lacZ promoters, the T3 and T7 promoters, the gpt promoter, the phage λ PR and PL promoters, the phoA promoter and the trp promoter. Eukaryotic promoters include the CMV (cytomegalovirus) immediate early promoter, the HSV (herpes simplex virus) thymidine kinase promoter, the early and late SV40 promoters and the promoters of retroviral LTRs (long terminal repeats). Other suitable promoters will be known to the skilled artisan. Expression vectors will desirably also contain sites for transcription initiation and termination, and in the transcribed region, a ribosome binding site for translation (see, for example, WO 98/16643)

Many expression systems are known, including systems employing: bacteria (e.g. E . coli and Bacillus subtilis) transformed with, for example, recombinant bacteriophage, plasmid or cosmid DNA expression vectors; yeasts (e.g. Saccharomyces cerevisiae) transformed with, for example, yeast expression vectors; insect cell systems transformed with, for example, viral expression vectors (e.g. baculovirus); plant cell systems transfected with, for example viral or bacterial expression vectors; animal cell systems transfected with, for example, adenovirus expression vectors.

The vectors can include a prolcaryotic replicon, such as the Col E1 ori, for propagation in a prokalyote, even if the vector is to be used for expression in other, non-prokaryotic cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prolcaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable maskers HIS3, TRP1, LEU2 and URA3. Plasmids pRS413-416 are Yeast Centromere plasmids (YCps).

Methods well mown to those skilled in the art can be used to construct expression vectors containing the coding sequence and, for example appropriate transcriptional or translational controls. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal Trough complementary base pairing and remaining nicks can be closed by the action of DNA ligase.

A further method uses synthetic molecules called linkers and adaptors. DNA fragments with blunt ends are generated by bacteriophage T4 DNA polymerase or E. coli DNA polymerase I which remove protruding 3′ termini and fill in recessed 3′ ends. Synthetic liners, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end.

More preferably, the present invention provides a use wherein the vector is suitable for replication in a mammalian cell.

By “suitable for replication” we include the meaning that the vector can be copied during replication and/or division of a mammalian cell such that the vector is transferred to the daughter cell(s) generated by replication and/or division of the parent mammalian cell.

Conveniently, the present invention provides a use wherein the vector is a viral vector, especially an adeno-associated viral vector.

By “adeno-associated viral vector” we include recombinant adeno-associated viral vectors (rAAV) derived from non-pathogenic viruses of the Parvoviridae family. Such viral vectors are icosahedral, 20-25 nm in diameter and have a single-stranded DNA genome. Replication of AAV is dependent on the presence of wild type adenovirus or herpes virus. In the absence of helper virus, AAV will stably integrate into the host cell genome whilst co-infection with helper virus triggers the lytic cycle.

It is well known that adeno-associated (AAV) vectors can efficiently transfer genes of interest to a broad range of mammalian cell types leading to high levels of stable and long-term expression after a single application. Importantly, they lack immunogenicity and have no known pathogenicity and have consequently been widely used in gene transfer approaches in experimental and clinical settings. The gene of interest is cloned into a plasmid vector, flanked by AAV inverted terminal repeat sequences. Suitable vectors include the rAVE expression plasmid available from Genedetect (www.genedetect.com), or the pAAV-MCS or pAAV-IRES-hrGFP vectors available from Stratagene (La Jolla, Calif. 92037, USA). In the presence of helper virus and AAV rep and cap genes (which are often provided on a separate plasmid vector) host cells produce recombinant AAV that includes the gene of interest in its genome.

The preferred viral vector is adeno-associated virus because of its well-documented safety and efficacy and because it has been shown to target retinal ganglion cells (RGCs) upon intra-vitreal injection. It will be understood by those skilled in the art that other viral vectors, such as adenovirus, lentivirus or herpesvirus, could also be used within the method of the invention.

Preferably, the present invention provides a use wherein the nucleic acid encodes a photoactivator and one or more proteins involved in the photo-transduction cascade.

For example, a nucleotide sequence encoding a protein that regulates retinoid metabolism may be introduced. Alternatively, a nucleotide sequence encoding a protein involved in the generation, amplification, transmission and termination of al intracellular signal following activation of a photoactivator in response to light stimulation.

In a further aspect, the present invention provides a vector comprising a nucleic acid encoding a photoactivator.

More preferably, the present invention provides a vector wherein the photoactivator is an opsin.

More preferably, the invention provides a vector wherein the photoactivator is an opsin and one or more co-factor of the opsin.

More preferably, the invention provides a vector wherein the photo activator is a mammalian opsin.

More preferably, the invention provides a vector wherein the photoactivator is a human opsin.

More preferably, the invention provides a vector wherein the photoactivator is melanopsin.

More preferably, the invention provides a vector wherein the nucleic acid has the nucleotide sequence of SEQ ID NO:1.

More preferably, the invention provides a vector wherein the vector is an expression vector.

More preferably, the invention provides a vector wherein the vector is suitable for replication in a mammalian cell.

More preferably, the invention provides a vector wherein the rector is a viral vector.

More preferably, the invention provides a vector wherein the viral vector is an adeno-associated viral vector.

In a further aspect, the present invention provides a neuronal cell comprising a nucleic acid according to the invention and/or a vector according to the invention, wherein the neuronal cell is capable of expressing a photo activator.

In a further aspect, the present invention provides a neuronal cell comprising a photoactivator according to the invention.

Preferably the neuronal cell is a retinal ganglion cell (RGC).

Preferably, the present invention provides a use of a photoactivator according to the invention and/or a nucleic acid according to the invention and/or a vector according to the invention and/or a neuronal cell according to the invention in the manufacture of a medicament for inducing photosensitivity in one or more neuronal cell an individual, wherein the photoactivator is capable of activating a photo-transduction cascade in a neuronal cell in response to light.

Preferably, the present invention provides a use wherein the neuronal cell is a retinal ganglion cell (RGC).

Preferably, the present invention provides a use of a photoactivator according to the invention and/or a nucleic acid according to the invention and/or a vector according to the invention and/or a neuronal cell according to the invention in the manufacture of a medicament for restoring sight. and/or alleviating blindness in all individual, wherein the photoactivator is capable of activating a photo-transduction cascade in a retinal ganglion cell (RGC) in response to light.

By “restoring sight and/or alleviating blindness” we include the result of restoring a degree of functional light responsiveness to retinal ganglion cells.

In humans, the methods for measuring and/or detecting this includes using conventional electro-diagnostic measures of Visually Evoked Potentials (VEPs) at the occipital pole. VEP is a clinical and/or research method of measuring the visual response that arrives at the visual cortex.

In addition, psychophysical measures of light sensitivity may be used.

It is known that in the normal retina, the network of neurones contributes to the spatial processing of visual signals. Whilst restoration of light responsiveness may be achieved using the present invention, it will be understood that the loss of visual processing that natural arises from the loss of the retinal network may also need to be considered. The degree of loss and non-linearity in the retinotopic mapping of RGC may be partially compensated by a degree of exterior processing through a prosthetic device with an optical output.

In particular, the present invention provides a use wherein the individual has a reduced number of functional photosensitive rod and/or cone cells in the eye.

Loss of functional photosensitive rod and/or cone cells in the eye may arise in several ways. Loss of functional rod and/or cone cells may be occur for example by: light damage; age related macular degeneration: or disease.

Preferably, the present invention provides a use wherein the individual has a reduced number of functional photosensitive rod and/or cone cells in the eye due to a condition selected from light damage, age related macular degeneration, or disease.

More preferably, the present invention provides a use wherein the disease is retinitis pigmentosa.

Retinal degenerations are the commonest cause of blindness in the Western world where over 1 in 20 of the population will develop retinal degeneration at some, stage in their lifetime. Clinically, afflicted individuals may be divided into two major groups: those with retinitis pigmentosa (in which the major pathology is initially in the peripheral rods) and those afflicted with macular degeneration (in which the brunt of the pathology is initially born by the central cones).

Retinitis pigmentosa is a hereditary disease and is estimated to affect 1 in 10,000 people. Age-related macular degeneration is estimated to affect 15 million people (Fine et al., 2000). In individuals with retinitis pigmentosa, loss of sight aid/or blindness may occur by progressive visual loss, which generally begins with rod loss and later cone degeneration. Late stage cases have no response to light which is thought to be associated with the total abolition of rod and cone photoreceptors.

Preferably, the present invention provides a use wherein the individual is human. The present invention may also provide a use wherein the individual is an animal, particularly animals such as dogs which are known to be affected by conditions that result in loss of functional rod and/or cone cells.

In a further aspect, the present invention provides a use of a nucleic acid according to the earlier aspects of the invention and/or a photoactivator according to the earlier aspects of the invention in the manufacture of a medicament for inducing photosensitivity in one or more retinal ganglion cell (RGC) in an individual, wherein the photoactivator is capable of activating a photo-transduction cascade in a retinal ganglion cell in response to light.

Preferably, the present invention provides a use wherein the medicament further comprises a pharmaceutically acceptable recipient, diluent or carrier.

Preferably, the present invention provides a use wherein the medicament is in a form adapted for delivery into the vitreal space. Preferably, the present invention provides a use wherein the medicament is in a form which is compatible with the vitreous humor/vitreous body. The way in which a medicament may be adapted for this form of delivery will be well known by those skilled in the relevant art.

In a further aspect, the present invention provides a pharmaceutical composition comprising a photoactivator according to the invention and/or a nucleic acid according to the invention and/or a vector according to the invention and/or a neuronal cell according to the invention, and a pharmaceutically acceptable carrier or exipient, the photoactivator and/or the nucleic acid and/or the vector and/or the neuronal cell being present in an amount effective to induce photosensitivity in one or more neuronal cell in an individual.

Preferably, the invention provides a pharmaceutical composition wherein the neuronal cell is a retinal ganglion cell (RGC).

In a further aspect, the present invention provides a pharmaceutical composition comprising a photoactivator according to the invention and/or a nucleic acid according to the invention and/or a vector according to the invention and/or a retinal ganglion cell (RGC) according to the invention. and a pharmaceutically acceptable carrier or exipient, the photoactivator and/or the nucleic acid and/or the vector and/or the retinal ganglion cell (RGC) being present in an amount effective to restore sight and/or alleviate blindness in an individual.

By “effective amount” we include an amount that is sufficient to induce photosensitivity in one or more RGCs and thereby restore sight and/or alleviate blindness in an individual. An effective amount may be determined by use of the methods described above for measuring and/or detecting whether sight has been restored and/or blindness has been alleviated. Alternatively, an idea of the effective range of a medicament may be obtained by testing the medicament on RGCs in vitro.

In humans, the methods for measuring and/or detecting this includes using conventional electro-diagnostic measures of VEPs at the occipital pole. Furthermore, we would use psychophysical measures of light sensitivity. Whilst restoration of light responsiveness is the direct goal, it will be understood that the loss of visual processing that naturally arises from the loss of the retinal network may also need to be considered. The degree of loss and non-linearity in the retinotopic mapping of RGC may be partially compensated by a degree of exterior processing through a prosthetic device with an optical output.

In a further aspect, the present invention provides a method of inducing photosensitivity in one or more neuronal cell comprising inducing the production of a photoactivator that is capable of activating a photo-transduction cascade in a neuronal cell in response to light.

Preferably, the invention provides a method wherein production of a photoactivator is induced in one or more neuronal cell by expressing a nucleic acid encoding a photoactivator in one or more neuronal cell.

By “inducing the production of a photoactivator” we include any process that results in the production and/or formation of a photoactivator within the cell. Many such processes are currently known in the art for inducing the production of molecules within cells, for example: the introduction and expression of a nucleic acid encoding a desired protein into a cell; the induction of expression of a nucleic acid that is usually present in the cell of interest but that is not usually expressed by that cell; the production of a desired molecule outside of the cell of interest using techniques known in the art and the introduction of that molecule into the cell of interest.

More preferably, the invention provides a method wherein the nucleic acid encoding a photoactivator is introduced into one or more neuronal cell. Introducing nucleic acids into appropriate cell hosts can be accomplished by well known methods that typically depend on the type of vector used. With regard to transformation of prokaryotic host cells, see, for example, Cohen et al (1972) and Sambrook et al (2001). Transformation of yeast cells is described in Sherman et al (1986). The method of Beggs (1979) is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Physical methods may be used for introducing DNA into animal and plant cells. For example, microinjection uses a very fine pipette to inject DNA molecules directly into the nucleus of the cells to be transformed. Another example Involves bombardment of the cells within high-velocity micro-projectiles, usually particles of gold or tungsten that have been coated with DNA. Alternatively, plasmids may be introduced into animal cells by electroporation or by liposome delivery (Matsuda et al., 2004).

Successfully transformed cells, i.e. cells that contain a DNA construct of the present invention, can be identified by well known techniques. For example, one selection technique involves incorporating into the expression vector a DNA sequence (marker) that codes for a selectable trait in the transformed cell. These markers include dihydrofolate reductase, G418 or neomycin resistance for eukaryotic cell culture, and tetracyclin, kanamycin or ampicillin resistance genes for culturing in Escherichia coli (E. coli) and other bacteria. Alternatively, the gene for such selectable trait can be on another vector, which is used to co-transform the desired host cell.

The marker gene can be used to identify transformants but it is desirable to determine which of the cells contain recombinant DNA molecules and which contain self-ligated vector molecules. This can be achieved by using a cloning vector where insertion of a DNA fragment destroys the integrity of one of the genes present on the molecule. Recombinants can therefore be identified because of loss of function of that gene.

Another method of identifying successfully transformed cells involves growing the cells resulting from the introduction of an expression construct of the present invention to produce a polypeptide photoactivator of the invention. Cells can be harvested and lysed and their DNA content examined for the presence of the DNA using a method such as that described by Southern (1975) or Berent et al (1985). Alternatively, the presence of the protein in the supernatant can be detected using antibodies as described below.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells. suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium.

More preferably, the invention provides a method wherein the nucleic acid encoding a photoactivator is usually present in the genome of a neuronal cell.

By “usually present in the genome of a retinal ganglion cell” we include the meaning that the nucleic acid encoding a photoactivator is part of the Genetic material (i.e. DNA and/or RNA) usually, contained in a neuronal cell. It is well known that the genome of a cell may contain genes that are not expressed (for example, due to transcriptional or translational silencing of the gene), and the present invention provides a method wherein a nucleic acid (encoding a photoactivator) that is present in the genome of a neuronal cell but that is not expressed, is expressed to induce photosensitivity in one or more neuronal cell.

More preferably, the invention provides a method wherein the neuronal cell is a retinal ganglion cell (RGC).

In a further aspect, the present invention provides a method of restoring sight and/or alleviating blindness in an individual comprising inducing the production of a photoactivator in one or more retinal ganglion cell (RGC) that is capable of activating a photo-transduction cascade in a retinal ganglion cell (RGC) in response to light.

In a further aspect, the present invention provides a method of inducing photosensitivity in one or more neuronal cell, the method comprising administering an effective amount of a medicament according to the invention and/or a pharmaceutical composition according to the invention to a subject in need thereof.

In a further aspect, the present invention provides a method of inducing photosensitivity in one or more neuronal cell in vitro, the method comprising introducing an effective amount of a photoactivator according to the invention and/or a nucleic acid according to the invention and/or a vector according to the invention into one or more neuronal cell.

It will be understood that the method of the invention includes the method of inducing photosensitivity in one or more retinal ganglion cell in vivo (for example, in an individual) and/or ex vivo (for example, outside the body of an individual) and/or in vitro (for example, in a cell culture).

Preferably, the present invention provides a method wherein the neuronal cell is a retinal ganglion cell (RGC).

In a further aspect, the present invention provides a method of restoring sight and/or alleviating blindness in an individual, the method comprising administering an effective amount of a medicament according to the invention and/or a pharmaceutical composition according to the invention to a subject in need thereof.

In a further aspect, the present invention provides a method of inducing photosensitivity in one or more neuronal cell of an individual, the method comprising the steps of:

(i) introducing a photoactivator according to the invention and/or a nucleic acid according to the invention and/or a vector according to the invention into one or more neuronal cell in vitro so that photosensitivity is induced in the one or more neuronal cell; and

(ii) administering an effective amount of the resulting one or more neuronal cell in which photosensitivity has been induced into a suitable site in the individual.

In a further aspect, the present invention provides a method of restoring sight and/or alleviating blindness in an individual, the method comprising the steps of:

(i) introducing a photoactivator according to the invention and/or a nucleic acid according to the invention and/or a vector according to the invention into one or more retinal ganglion cell (RGC) in vitro so that photosensitivity is induced in the one or more retinal ganglion cell (RGC); and

(ii) administering an effective amount of the resulting one or more retinal ganglion cell (RGC) in which photosensitivity has been induced into one or both eye(s) of an individual.

In a further aspect, the present invention provides a method of inducing photosensitivity in one or more neuronal cell in an individual, the method comprising the step of administering an effective amount of one or more neuronal cell according to the invention to an individual.

In a further aspect, the present invention provides a method of restoring sight and/or alleviating blindness in an individual, the method comprising the step of administering an effective amount of one or more retinal ganglion cell (RGC) according to the invention to an individual.

Preferred, non-limiting examples which embody certain aspects of the invention will now be described, with reference to the following figures:

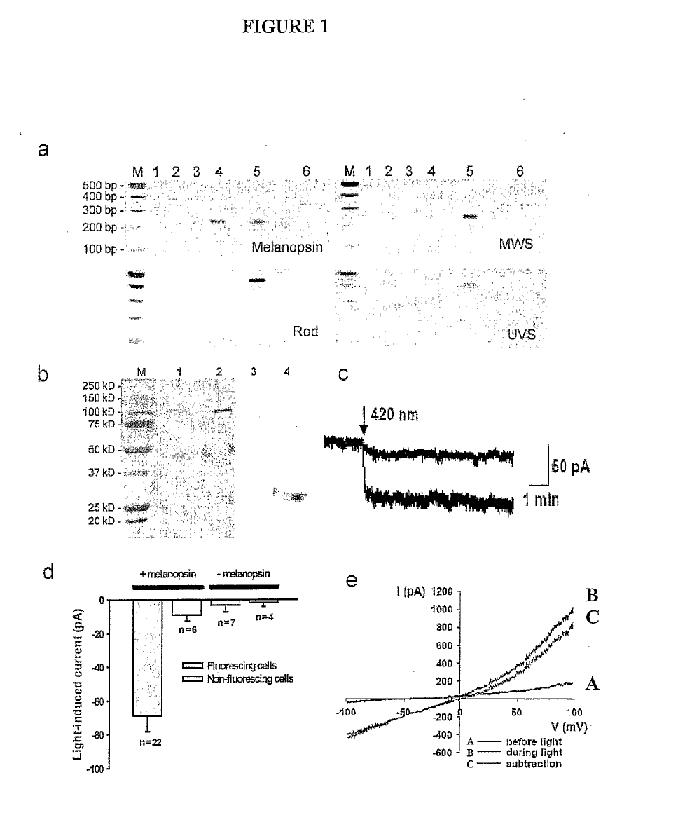

FIG. 1 Expression of human melanopsin renders Neuro-2a cells light sensitive. (a): RT-PCR (reverse transcription polymerase chain reaction) analysis demonstrated that neither undifferentiated (lane 1) nor differentiated (lane 2) Neuro-2a cells expressed melanopsin or murine rod or cone (MWS or UVS) opsins in their native state. Transfection with a control plasmid (pCMS-EGFP) lacking human melanopsin did not change this expression pattern (lane 3), while transfection with the melanopsin expression vector successfully induced melanopsin expression but not any other opsin (lane 4). Lane 5 is a positive control (mouse retinal cDNA) and lane 6 a no template control. (b): Western blot of protein extracted from Neuro-2a cells transfected with expression vector driving C-terminal 6×His tagged human melanopsin and EGFP (lanes 2 and 4) or mock-transfected (lanes 1 and 3). Hybridisation with an anti-tetra-His antibody (lanes 1 and 2) revealed a single specific band at around 50 kD (with reference to size standards; lane M) in the transfected cells corresponding to the expression of human melanopsin (expected size 53.5 kD). Expression of EGFP in these cells was also reflected in a single band at around 28 kD (expected size 26.9 kD) following hybridization with anti-EGFP antibody (lanes 3 and 4). (c): Whole cell recordings from a melanopsin transfected cell exposed (shown by arrow) to a 420 nm, 10 sec light stimulus at either 8×10¹³ (top trace) or 8×10¹⁴ photons/cm²/sec (bottom trace) revealed an intensity dependent sustained inward current. (d): Light evoked currents are melanopsin dependent. Fluorescent cells successfully transfected with melanopsin-EGFP and exposed to 9-cis retinal showed prominent inward currents. Small residual currents were seen in neighboring non-fluorescent cells, consistent with a low level expression of the vector. Parallel recordings from the EGFP-alone transfected cells revealed no significant light evoked currents irrespective of transfection status. (e): Current-voltage relationship of the light-activated current in a melanopsin expressing cell exposed to 9-cis retinal. The light-activated current (red; line C) was determined by subtracting currents elicited by the voltage ramp (−100 to +100 mV over 2s) before (black; line A) and during (blue; line D) illumination. The resulting light-activated current was consistently inward and linear between −100 and −50 mV with a population slope conductance of 6.46±0.82 nS (mean±SEM, n=13).

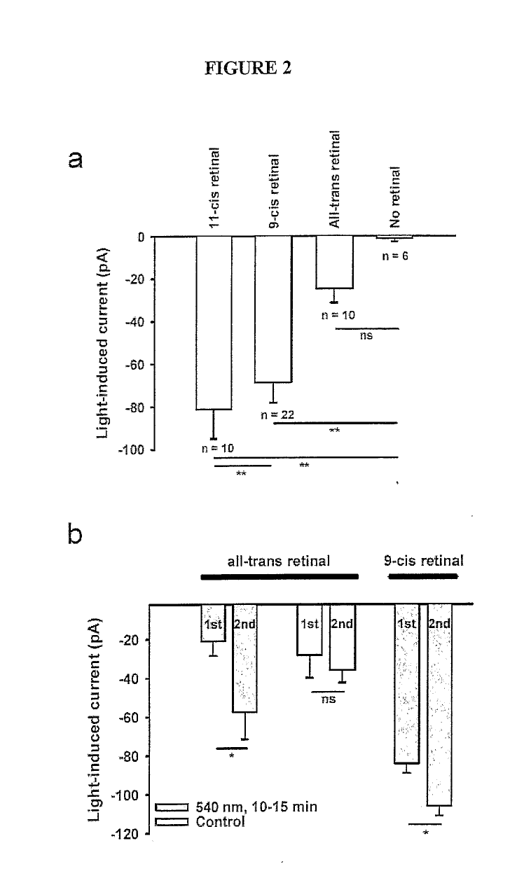

FIG. 2. (a): Retinaldehyde dependence of the light evoked current. Comparison of light (420 mn 8×10¹⁴ photons/cm²/sec stimulus) evoked currents in melanopsin expressing cells preloaded with 11-cis-retinal, 9-cis-retinal or all-trans-retinal (all 20 μM, 1 hour) and those with no preloading. One-way “analysis of variance” (ANOVA) revealed an effect of treatment (P<0.0001) on the peal inward current observed, with both 11-cis and 9-cis-retinal groups more responsive p<0.01) than the other two. (b) Exposure to long-wavelength light selectively enhances the light evoked current in cells that are preloaded with all-trans-retinal. Cells were loaded with all-trans retinal (20 μM) and the inward current measured in response to light (420 nm, 10 sec, 8×10¹⁴ photons/cm²/s). The cells recovered for 10 mins and were then exposed for 10 mins to light of 540 nm (2×10¹⁴ photons/cm²/s). The cells were then retested with the 420 nm stimulus (n=5). Paired t-tests revealed a large increase in inward current following the 540nm light exposure in all-trans (<0.05) and, to a lesser extent, 9-cis (p<0.05) loaded cells, which was not observed in the absence of the 540 nm stimulus (Control; p>0.05).

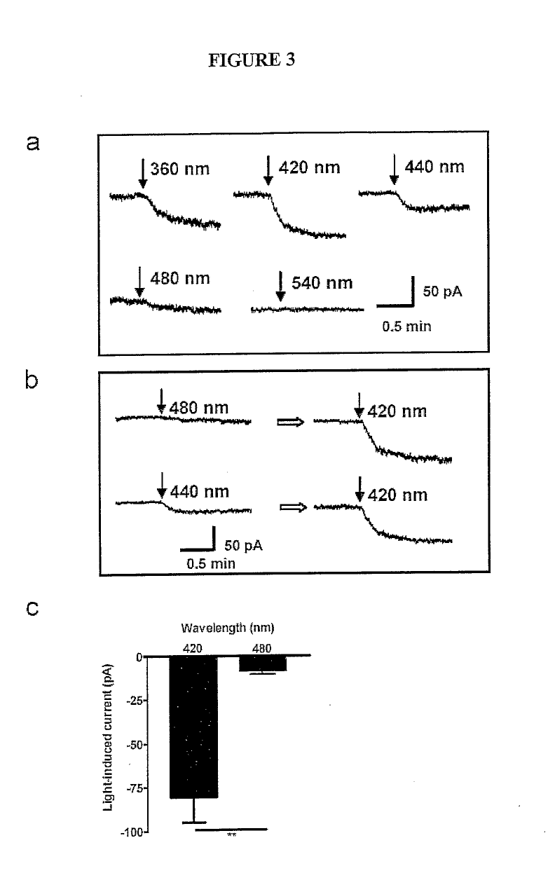

FIG. 3. Spectral sensitivity of the light evoked current (a): Representative responses to 360, 420, 440, 480 and 540 nm stimuli (at 3×10¹⁴, 8×10¹⁴, 2×10¹⁵, 3×10¹⁵ and 2×10¹⁴ photons/cm²/s respectively). The 360 and 420 nm stimuli gave equivalent responses (mean±SEM 72.5±19 and 80±11 pA (pico-amps) respectively) while responses to the longer wavelengths became progressively reduced; 32.7±14 pA at 440 nm, 8.5±1.2 pA at 490 nm and 2.0±1.2 pA at 540 nm. (b); Pairwise comparisons in the same cell between responses to 420 nm and 440 or 480 nm also indicated that the shorter wavelength was more effective. (c); A direct comparison between paired 480 and 420 nm responses in 7 different cells confirmed this finding (paired t-test, p<0.01).

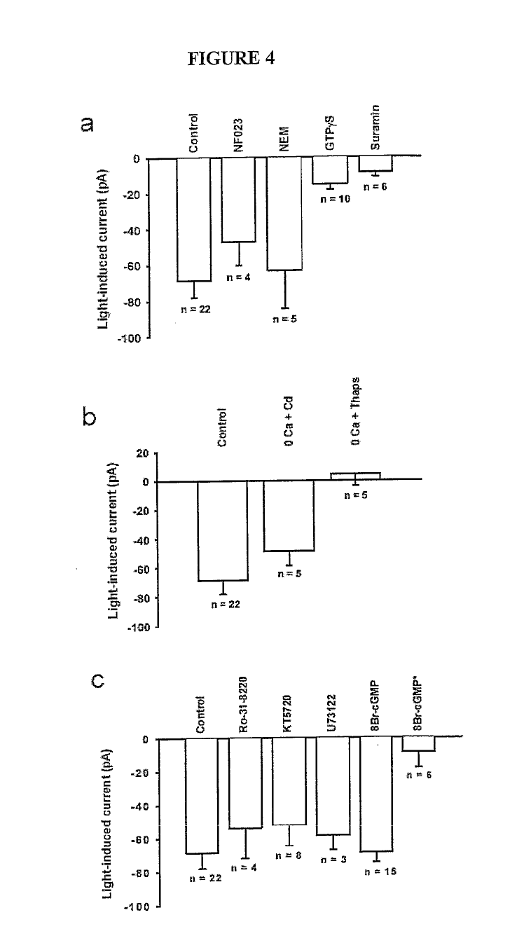

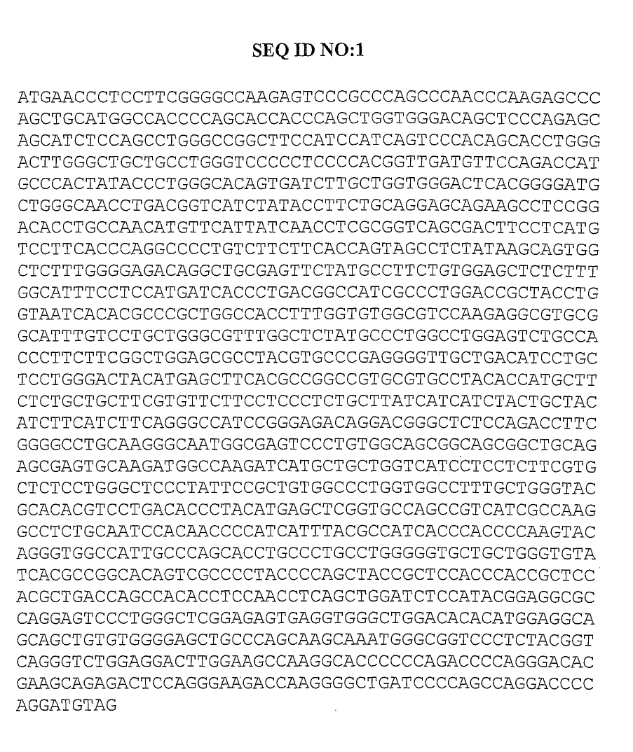

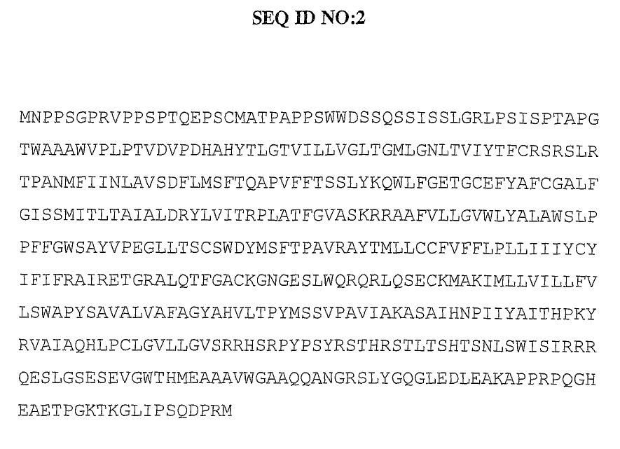

FIG. 4. Properties of the melanopsin transduction cascade. (a); G protein coupling of the response. NF023 (up to 1 μM) and NEM (50 μM) applied in the bath had no effect on light evoked currents. Whilst either GTPγS (1 mM: in patch pipette) or suramin (100 μM; in bath) largely abolished photoresponses. (b); calcium signaling. Removal of calcium from the perfusate and/or the addition of Cd²⁺ (100 μM) had little significant effect on the light evoked current. Whilst treatment with thapsigargin (5 to 10 μM) in zero calcium perfusates for 15-20 mins abolished the light evoked current. In each case the light evoked currents were evoked in response to a 420 nm, 10 sec stimulus (8×10¹⁴ photons/cm²/s) and the cells were preloaded with 9-cis retinal (20 μM). (c); Second messengers. RO31-8220 (100 nM) KT5720 (1 μM) U73122 (50 μM) did not affect light induced current, whilst including 8-Br-cGMP in the patch pipette caused a dose dependent suppression of the current (8-Br-cGMP=100 μM, 8-Dr-cGMP*=1 nM). In each case the light evoked currents were evoked in response to a 420 nm, 10 sec stimulus (8×10¹⁴ photons/cm²/s) and the cells were preloaded with 9-cis retinal (20 μM).

### EXAMPLE 1

### Experimental Data Demonstrating Induction of Photosensitivity in a Culture Neural Cell Line

### Methods

### Generation of Constructs.

The full length coding sequence of human melanopsin was amplified from retinal cDNA using Pfu-Turbo DNA polymerase (Stratagene) and the following restriction tagged primers:

HMELF: 5′-ccggaattcatcccaactcaggatgaacc-3′,
and;
HMELR: 5′-tgctctagacgtcctacatcctggggtc-3′.

The product was cloned into the pLITMUS28 Vector (New England Biolabs) and sequenced to confirm that there was no deviation from the reported sequence NM_{—}033282; SEQ ID NO: 1). The product was subsequently cloned into the pCMS-EGFP vector (BD Biosciences), such that expression would be driven by the CMV immediate early promoter. A C-terminal 6×His-tagged melanopsin construct was generated in a similar manner, using the following primers:

OPN4/5′: 5′-gaagatctcatcccaactcaggatgaaccctc-3′,
and;
OPN4/3′PH: 5′-cggaattcctagtgatggtgatggtgatgcatcctg
gggtcctggctggggatcag-3′.

For analyses of spectral sensitivity EGFP expression was excluded by employing the pCI-neo mammalian expression vector (Promega).

### Cell Culture and Intracellular Recordings.

Mouse Neuro-2a cells (American Type Culture Collection; Cat no. CCL-131) were maintained at 37° C. in D-MEM, Dulbecco's Modified Eagle Medium with GlutaMAX™ I, 4500 mg/l D-Glucose, Sodium Pyruvate (D-MEM, Invitrogen) with 10% foetal bovine serum, 1% non-essential amino acids and 20 μg/ml gentomycin in a 5% CO₂ atmosphere. Cells were trypsintsed, split and cultured at 1×10⁵/ml in 90 mm dishes for 24 hours prior to transfection with plasmid vectors using lipofectamine (Invitrogen) in serum-free medium. DNA to lipid ratio was 10 μg DNA: 100 μg lipid. After transfection serum free medium was replaced with normal medium. On the following day, 2-3 days before study, cell differentiation was induced using 10 μM retinoic acid (Provencio et al., 2000). Starting from this point cells were kept in darkness. On the day of recording, glass coverslips serving as a substrate for cell attachment were transferred to the recording chamber. The perfusion solution contained 140 mM NaCl, 4 ml KCl, 1 mM MgCl₂, 2 mM CaCl₂, 5 mM glucose, 10 mM HEPES (pH 7.3-7.4; 24° C.). Successfully transfected cells were identified by EGFP fluorescence under a 480 nm stimulus light using an OLYMPUS BX51WI microscope. At this point, retinal isoforms (20 μM; 9-cis and all-trans from Sigma-Aldrich; 11-cis from Dr R K Crouch, Medical University of S.C.) were added to the perfusion solution as necessary and cells kept in the dark for at least 1 hour before recording. Whole-cell patch-clamp recordings were made with pipettes containing 140 mM KCl, 10 mM NaCl, 1 mM MgCl₂, 10 mM HEPES, 10 mM EGTA. Osmolarity was adjusted to 285±5 mosmol (milliosmol) 1⁻¹ and pH to 7.3-7.4 with KOH. Open pipette resistance was 2-5 MΩ (mega-ohms), and access resistance during recordings was <20 MΩ. Currents were recorded (Axopatch 200B, Axon Instruments) in neurons voltage clamped at holding potentials of −50 mV (milli-volt). The records were filtered at 1 kHz (kilohertz) and sampled at 20 kHz. Drugs were obtained from Sigma Aldrich, with the exception of NEM (N-ethylmaledomide) (Calbiochem), and DL-AP5, NF023, KT5720, U73122 and thapsigargin (all Tocris Cookson), drugs applied in superfusion were applied for 15-20 minutes prior to light stimulation.

Light stimuli were generated using a Cairn Optoscan Xenon arc source comprising a slit monochromator. Unless otherwise stated al stimuli were 10 sec in duration with a 20 nm half-bandwidth. Irradiance was measured using an optical powermeter (Macam Photometrics) and converted to photon flux. It has been suggested that NMDA (N-methyl-D-aspartate) receptors may include a light sensitive moiety. The nature of glutamate receptor expression in Neuro-2a cells remains ambiguous, nonetheless we excluded the possibility that this might be the origin of the melanopsin light response by application of the selective NMDA antagonist APV (2-amino-5-phosphonovaleric acid) (100 μM) which had no effect upon the light sensitive current (data not shown)

### Opsin Expression in Neuro-2a Cells by RT-PCR.

Cells were harvested both before and after differentiation, and following transfection with pCMS-EGFP vector alone, or with the human melanopsin-EGFP vector. RNA Alas extracted using Tri reagent (Sigma), and treated with DNaseI (Promega) prior to reverse transcription. Single-stranded cDNA was synthesised using the SuperScript First-Strand Synthesis System for RT-PCR (Invitrogen). Specific primers for melanopsin, rod opsin, UVS and MWS-cone opsins, and RGR-opsin and peropsin were used. as well as primers for the rod and cone transducin alpha subunits (Gnat1 and Gnat2). The absence of vector DNA carry over in both of the transfected Neuro-2a cDNA samples was confirmed using primers designed to the cytomegalovirus (CMV) promoter (data not shown).

The primers used are as follows:

Rod opsin:
RodF: 5′-tcaagccggaggtcaacaac-3′
RodR: 5′-tcttggacacggtagcagag-3′
UVS opsin:
UVSf: 5′-cagccttcatgggatttg-3′
UVSr: 5′-gtgcatgcttggagttga-3′
MWS opsin:
MWSf1: 5′-caagcatcttcacctatacc-3′
MWSr1: 5′-cgctgaacacgtctgggc-3′
Melanopsin:
Melf: 5′-atcctgctcctgggactac-3′
Melr: 5′-tcttggccatcttgcactc-3′
Peropsin:
PerF: 5′-cctgatgtaggacgaagaatgacc-3′
PerR: 5′-cacaagcacacgatggaataagg-3′
RGR-opsin:
mRGR F: 5′-gaggggtgacagaaacttcatcag-3′
mRGR R: 5′-ttgtggagacagacactgccag-3′
GNAT1 (rod transducin alpha subunit):
Gnat1f: 5′-gagaagcactccagagagc-3′
Gnat1r: 5′-ttgagctggtattctgagg-3′
GNAT2 (cone transducin alpha subunit):
Gnat2f: 5′-agtggcatcagtgctgagg-3′
Gnat2r: 5′-cgagtcattgagctggaac-3′

### Western Blots.

Protein was extracted from both mock-transfected Neuro-9 a cells and from cells transfected with the C-terminal H is-tagged melanopsin-EGFP construct using Tris buffer containing 15% SDS, 100 mM DTT and protease inhibitors. After quantification using a standard protein assay, kit (Sigma); equal quantities of protein were loaded on 10-20% linear gradient Tris-HCl polyacrylamide gels (Bio-Rad), and subsequently blotted onto PVDF membrane (Amersham Biosciences). C-terminal His-tag melanopsin was detected using a Tetra-His antibody (QIAGEN) at 1:1000 dilution, and EGFP protein using the mAB11E5 antibody (Qbiogene) at a 1:1000 dilution. The secondary antibody was polyclonal goat anti-mouse/HRP (DakoCytomation) at 1:1000 dilution. Detection was carried out by enhanced chemiluminescence (Amersham Biosciences).

### Results

The inventors set out to determine the function of melanopsin in an intact cellular environment by examining the ability of heterologous expression of human melanopsin to render mammalian neuronal cells photoreceptive. The mouse neuroblastoma cell line (Neuro-2a) was chosen for this purpose, because of its proven ability to support exogenous G-protein based signalling (Spencer et al., 1997). Neuro-2a cells were transfected with plasmid expression vectors based upon the pCMS-EGFP plasmid (BD Biosciences) using the lipofectamine method and differentiated with retinoic acid (Shea et al., 1985). The vectors were engineered to express the complete coding sequence of human melanopsin under control of the immediate early promoter of cytomegalovirus (CMV), along with an enhanced green fluorescent protein (EGFP) reporter gene driven by an SV40 enhancer/promoter. A C-terminal hexa-histidine (6×His) tag was added to the melanopsin sequence as required to facilitate detection of the protein. We found no discernable difference in the biological effects of the tagged and un-tagged proteins.

High transfection efficiencies were obtained using these techniques, with up to 60% of Neuro-2a cells expressing EGFP as assessed by fluorescence microscopy. Untransfected Neuro-2a cells did not express melanopsin (or rod/cone opsins) in either their differentiated or undifferentiated states (FIG. 1). However, following plasmid transfection, human melanopsin could be observed at both the mRNA and protein level (FIG. 1).

Physiological light responses were first assessed following pre-incubation with 9-cis retinal (1 hour, 20 μM). Under these conditions, untransfected Neuro-2a cells were not photoresponsive (FIG. 1). Nor did transfection with an EGFP expression vector lacking melanopsin induce photosensitivity (FIG. 1). However, expression of human melanopsin was sufficient to produce a marked cellular response to light exposure. A 10 sec stimulus of 420 nm (20 nm half bandwidth) light resulted in a significant inward current, the magnitude of which was irradiance dependent (FIG. 1). Further analysis of the response to the brighter stimulus using holding voltage ramps revealed a peak light evoked slope conductance of 6.46±0.82 nS (mean±SEM (standard error of mean), n=13), some three times greater than that previously observed in ipRGCs following exposure to a similar light intensity (Warren et al., 2003).

The ability of human melanopsin to render Neuro-2a cells photoreceptive suggests that, under these circumstances, this protein can form a sensory photopigment. All opsin photopigments employ retinal as a light absorbing chromophore, so we continued to assess the retinal dependence of the melanopsin effect. The photoreceptive function of melanopsin was indeed dependent upon preincubation with an appropriate isofrom of retinal (FIG. 2). In the absence of exogenous retinal, melanopsin transfected cells were not light sensitive. Significant photosensitivity was observed following preincubation with either 9-cis or 11-cis retinal. 11-cis was significantly more effective, probably reflecting the higher inherent photos sensitivity of opsin photopigments reconstituted with this isoform (Liu et al., 1986).

These findings are consistent with the hypothesis that melanopsin acts as a photopigment with a specific affinity for cis-isoforms of retinal. Other photopigments with such a photochemistry would not show photosensitivity following incubation with all-trans retinal. In fact, application of all-trans retinal in this preparation did seem to support a modest light response (FIG. 2), and, although statistical analysis did not confirm this trend, it seemed worthy of further investigation. Phylogenetically, melanopsin is most closely related to the cephalopod rhodopsins (Provencio et al., 1998), which use 11-cis retinal as a chromophore for sensory functions, but are also capable of binding all-trans retinal which they re-isomerise to 11-cis upon appropriate light exposure (Hubbard et al., 1958). Such an isomerase function would facilitate photosensitivity following incubation with all-trans retinal provided that the test stimulus was of sufficient duration to generate cis retinal in the first instance.

To examine the possibility that melanopsin has such an inherent chromophore regeneration capability, the effect of prior light exposure with longer wavelength light (540 nm, 20 nm half-bandwidth) on the photosensitivity of cells preincubated with 9-cis or all-trans retinal was examined. Stimulation with 540 nm light did not in itself evoke a light-induced response in either all-trans or 9-cis incubated cells (FIG. 3). However, pre-exposure to 540 nm light for 10 to 15 minutes did have a marked effect on subsequent light responses in cells loaded with all-trans retinal, typically resulting in a three-fold increase in light evoked currents in response to a 10 sec 420 nm stimulus (FIG. 2). No such increase was observed in cells held in the dark between stimuli. These data are consistent with the hypothesis that the 540 nm stimulus drives a photoisomerisation of all-trans to a cis isoform of retinal within these cells. In further support of this conclusion, our data show that the large magnitude effect of 540 nm light was specific to cells preloaded with all-trans retinal as cells loaded with 9-cis showed only a small potentiation. Interestingly, a small increase in photosensitivity in 9-cis loaded cells is also predicted by such a mechanism through the generation of the more sensitive 11-cis photopigment (FIG. 1) under these conditions.

We failed to find evidence that Neuro-2a cells express either of the known mammalian photoisomerases (RGR (retinal G-protein coupled receptor) and peropsin, data not shown), raising the possibility that melanopsin itself is providing this function. Thus, melanopsin may act as a photoisomerase in Neuro-2a cells. However, presentation of cis-retinal was not sufficient to induce photosensitivity in the absence of melanopsin. Consequently, such a function alone cannot explain the effects of human melanopsin expression in Neuro-2a cells. Rather, our findings support the hypothesis that melanopsin is a bistable pigment, similar to the related cephalopod. rhodopsins (Dixon et al., 1987), employing cis-isoforms of retinal in its photosensory function and acting as a photoisomerase to regenerate bleached chromophore. Such a photochemistry would have obvious advantages for a photopigment located in the inner retina distant from the retinal pigment epithelium, the primary site of chromophore regeneration, and indeed has been reported for a different vertebrate photopigment occupying a similar environment (Koyanagi et al., 2004).

Although direct measures of spectral sensitivity in human ipRGCs are unavailable, action spectrum studies have suggested peak sensitivity (lambdamax—λₘₐₓ) in the range 455-484 nm (Brainard et al., 2001; Thapan et al., 2001; Hankins et al., 2002). In order to assess the correspondence, if any, between the Neuro-2a light response and these action spectra we assessed cellular responses to stimuli ranging from 300 to 540 nm in cells transfected with a pCI-neo expression vector (Promega) driving human melanopsin. EGFP was not included as a marker of expression for these experiments because of its light absorbing and fluorescent properties. Instead, successful transfection was determined by a functional cellular response to the test wavelength or, where that proved ineffective, to a subsequent 420 nm stimulus. Cells that failed to show a robust physiological response to both of these stimuli were excluded from subsequent analysis. Cells were preincubated with 11-cis retinal, although similar results where obtained with 9-cis retinal as a chromophore (data not shown).

Stimuli at 360 and 420 nm were similarly effective at inducing a cellular response (FIG. 3). Responses at shorter wavelenghts were greatly reduced (data not shown) although the significance of this finding is uncertain because the available light at these wavelengths was also substantially attenuated. By contrast, responses to longer wavelengths were impaired despite being associated with increases in light intensity (FIG. 3), indicating a short wavelength shift in comparison with the human action spectra. In view of the potential for differing melanopsin expression levels between cells under transient transfection, this result was confirmed by direct comparison of responses to 420 and 480 nm light in the same cell. The shorter wavelength was clearly More effective in all of the cells tested (FIG. 3). We conclude that under these conditions human melanopsin has a λₘₐₓ in the range 360-430 nm.

Previous attempts to describe the spectral sensitivity of melanopsin, employing purified preparations of mouse melanopsin harvested from COS cells, have reported light absorbing complexes with λₘₐₓ around 420 or 440 nm depending upon light history (Newman et al., 2003). The data shown here suggest that the physiologically relevant isomerisation event for human melanopsin has a λₘₐₓ around 420 nm or shorter. The long wavelength sensitive photoreversal event described above (FIG. 2) may shift this λₘₐₓ to slightly longer wavelengths under examination with long duration stimuli, but this is unlikely to account for the full discrepancy with human action spectra. Of the remaining explanations, parsimony favours the hypothesis that some aspect of the intracellular environment of ipRGCs shifts the spectral sensitivity of melanopsin to longer wavelengths or screens shorter wavelength light. This might be brought about by a subtle change in the folding or post-translational modification of melanopsin. but the functional light response in Neuro-2a cells suggests that the structure of melanopsin in this environment is not grossly inappropriate.

Photosensory opsins in both vertebrate and invertebrate photoreceptors are G-protein coupled receptors. The mammalian melanopsins retain conserved structural features of G-protein coupled receptors (Provencio et al., 2000) and can interact with transducin in vitro (Newman et al., 2003). However, putative G-protein interaction domains are highly divergent among melanopsins from different vertebrate species (Bellingham et al., 2002). The importance of G-protein signalling for human melanopsin function was demonstrated in these experiments by the ability of either GTPγS (Gilman et al., 1984) (1 mM in patch pipette) or suramin (Beindl et al., 1996) (100 μM in the bath) to abolish light responses (FIG. 4a). However, human melanopsin did not appear to be coupled to the classical mammalian photoreceptor G-protein in the Neuro-2a cells as neither rod nor cone transducins were expressed in these cells (by RT-PCR, data not shown) and pharmacological block of Gi/Go pathways (NF023 Beindl et al., 1996) or N-ethyl (Asano et al., 1986)) did not antagonise the light response (FIG. 4).

As human melanopsin acts as a G-protein coupled receptor, the details of its intracellular transduction cascade are likely to be host-cell specific. In Neuro-2a cells, we found evidence for the involvement of both intracellular calcium mobilisation and cGMP in linking the receptor to a cell surface ion channel (FIG. 4). Thus, thapsigargin (5-10 μM, applied for 15-20 mins in bath) and 8-Br-cGMP (1 mM in pipette) abolished the light response. Specific antagonists of protein kinase A and C did not block the light response (FIG. 4). Nor did U73122 (effective against phospholipase C (Bleasdale et al., 1990) although, in view of the thapsigargin effect, the possibility that this drug has failed to reach the target site at an effective concentration must be considered. The nature of the ion channel regulated by melanopsin activity was investigated by replacement of sodium from the perfusate, removal of calcium and blockade of voltage gated calcium channels none of which significantly altered the light response (FIG. 4 and data not shown). In view of the sensitivity to thapsigargin and the reversal potential of the response (0 to −10 mV; FIG. 1), the most plausible candidate is a large-conductance calcium-activated anion channel expressed by Neuro-2a cells (Nobile et al., 2000).

### CONCLUSIONS

The results of these experiments have shown for the first time that expression of melanopsin is not only necessary (Lucas et al., 2003) but also sufficient to render cells functionally photoreceptive. They confirm the ability of human melanopsin to act as a functional sensory photopigment employing cis-isoforms of retinal and coupled to a G-protein signalling pathway. Importantly, the melanopsin photopigment also appears to have an intrinsic mechanism for the regeneration of bleached chromophore. These findings suggest a simple model for the photobiology of ipRGCs in which a single protein (melanopsin) subserves both sensory and photoisomerase functions. Under these circumstances, light would drive both photopigment bleach and bleach-recovery processes in a direct manner that has not previously been observed for a mammalian photoreceptor but is common in invertebrates (Hubbard et al., 1958). This could explain one of

The more surprising observations made of ipRGCs, their apparent resistance to bleach under sustained illumination (Berson et al., 2002).

An important aspect of this work is the demonstration that, in neuronal cells, a functional photoreceptor can be created by the introduction of only two components; human melanopsin and retinaldehyde. This raises the prospect of employing melanopsin expression to render cells photosensitive in a wide variety of experimental and, perhaps, therapeutic applications (Zemelman et al., 2002).

That we can engender photo-responsiveness in a neuronal cell line has a number of applied implications. The elegance of involving a single gene, together with a relative independence from a constant supply of active chromophore, suggests this may represent a unique way of rendering neurons light responsive. This approach may prove very valuable in restoring light responsiveness to neurones and also as tool a research tool in neuroscience.

For example, the introduction of a mammalian opsin into RGCs provides a means for restoring sight and/or alleviating blindness. This approach will transform the RGCs of a dystrophic retina into cells capable of both translating light exposure into a biological signal, and conveying that information to the visual centres of the brain and therefore provides a simple and reliable approach to the treatment of blindness.

### EXAMPLE 2

### Preferred Pharmaceutical Formulations and Modes and Doses of Administration

The nucleic acids, molecules and pharmaceutical formulations of the present invention may be delivered using an injectable sustained-release drug delivery system. These are designed specifically to reduce the frequency of injections. -A example of such a system is Nutropin Depot which encapsulates recombinant human grouch hormone (rhGH) in biodegradable microspheres that, once injected, release rhGH slowly over a sustained period.

The nucleic acids, molecules and pharmaceutical formulations of the present invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of nucleic acids, molecules and pharmaceutical formulations of the invention. A device which delivers a pulsed electric field to cells increases the permeability of the cell membranes to the drug, resulting in a sufficient enhancement of intracellular drug delivery.

The nucleic acids, molecules and pharmaceutical formulations of the invention can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of delivery of the nucleic acids, molecules and pharmaceutical formulations of the invention is the ReGel injectable system that is thermo-sensitive. Below body temperature, ReGel is an injectable liquid while at body temperature it immediately forms a gel reservoir that slowly erodes and dissolves into known, safe, biodegradable polymers. The active substance is delivered over time as the biopolymers dissolve.

The nucleic acids, molecules and pharmaceutical formulations of the invention can also be delivered orally. The process employs a natural process for oral uptake of vitamin B₁₂ in the body to co-deliver proteins and peptides. By riding the vitamin B₁₂ uptake system, the nucleic acids, molecules and pharmaceutical formulations of the invention can move through the intestinal wall. Complexes are synthesised between vitamin B₁₂ analogies and the drug that retain both significant affinity for intrinsic factor (IF) in the vitamin B₁₂ portion of the complex and significant bioactivity of the active substance of the complex.

The nucleic acids, molecules and pharmaceutical formulations of the invention can be introduced to cells by “Trojan peptides”. These are a class of polypeptides called penetrations which have translocating properties and are capable of carrying hydrophilic compounds across the plasma membrane. This system allows direct targeting of oligopeptides to the cytoplasm and nucleus, and may be non-cell type specific and highly efficient. See Derossi et al. (1998).

Preferably, the pharmaceutical formulation of the present invention is a unit dosage containing a daily dose or unit, daily sub-dose or an appropriate fraction thereof, of the active ingredient.

The nucleic acids, molecules and pharmaceutical formulations of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the nucleic acids, molecules and pharmaceutical formulations of the invention can be administered alone but will generally be administered in admixture with a suitable pharmaceutical excipient diluent or carrier selected with regard to the intended route of administration and standard pharmaceutical practice.

For example, the nucleic acids, molecules and pharmaceutical formulations of the invention can be administered orally, buccally or sublingually in the form of tablets, capsules, ovules, elixirs, solutions or suspensions, which may contain flavouring or colouring agents, for immediate-, delayed- or controlled-release applications. The nucleic acids, molecules and pharmaceutical formulations of the invention may also be administered via intracavernosal injection.

Such tablets may contain exipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar tape may also be employed as filters in gelatin capsules. Preferred exipients in this regard include lactose, starch, cellulose, milk sugar or high molecular weight polyethylene glycols. For aqueous suspensions and/or elixirs, the compounds of the invention may be combined with various sweetening or flavouring agents. colouring matter or dyes, with emulsifying and/or suspending agents and with diluents such as water, ethanol, propylene glycol and glycerin, and combinations thereof.

The nucleic acids, molecules and pharmaceutical formulations of the invention can also be administered parenterally, for example, intravenously, intra-arterially, intraperitoneally, intra-thecally, intraventricularly, intrasternally, intracraltially, intra-muscularly or subcutaneously, or they may be administered by imitation techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the hind previously described.

The nucleic acids and molecules and pharmaceutical formulations of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134AL or 1,1,1,2,3,3,3 heptafluoropropane (HFA 227EA,), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder nix of a compound of the invention and a suitable powder base such as lactose or starch.

Alternatively, the nucleic acids, molecules and pharmaceutical formulations of the invention can be administered in the form of a suppository or pessary, or they may be applied topically in the form of a lotion, solution, cream, ointment or dusting powder. The nucleic acids, molecules and pharmaceutical formulations of the invention may also be transdermally administered, for example, by the use of a skin patch. They may also be administered by the ocular route, particularly for treating diseases of the eye.

For ophthalmic use, the nucleic acids, molecules and pharmaceutical formulations of the invention can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the nucleic acids, molecules and pharmaceutical formulations of the invention can be formulated as a suitable ointment containing the active compound suspended or dissolved in, for example, a mixture with one or more of the following: mineral oil, liquid petrolatun, white petrolatum, propylene glycol, polyoxyethylene polyoxypropylene compound, emulsifying wax and water. Alternatively, they can be formulated as a. suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or parenteral administration of the nucleic acids, molecules and pharmaceutical formulations of the invention compounds of the invention is the preferred route, being the most convenient.

For veterinary use, the nucleic acids, molecules and pharmaceutical formulations of the invention is administered as a suitably acceptable formulation in accordance with normal veterinary practice and the veterinary surgeon will determine the dosing regimen and route of administration which will be most appropriate for a particular animal.

Conveniently, the formulation is a pharmaceutical formulation.

Advantageously, the formulation is a veterinary formulation.

### EXAMPLE 3

### Exemplary Pharmaceutical Formulations

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious 1 the recipients thereof Typically, the carriers will be water or saline which will be sterile and pyrogen-free.

The following examples illustrate pharmaceutical formulations according to the invention in which the active ingredient is a nucleic acid or molecule of the invention.

### Example A

### Tablet

| | | |
|---|---|---|
| Active ingredient | 100 | mg |
| Lactose | 200 | mg |
| Starch | 50 | mg |
| Polyvinylpyrrolidone | 5 | mg |
| Magnesium stearate | 4 | mg |
| | 359 | mg |

Tablets are prepared from the foregoing ingredients by wet granulation followed by compression.

### Example B

### Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |

| | |
|---|---|
| pH adjusted to | 7.5 |

### Example C

### Tablet Formulations

The following formulations A and B are prepared by wet granulation of the ingredients with a solution of povidone, followed by addition of magnesium stearate and compression.

Formulation A

| | mg/tablet | mg/tablet |
|---|---|---|
| (a) Active ingredient | 250 | 250 |
| (b) Lactose B.P. | 210 | 26 |
| (c) Povidone B.P. | 15 | 9 |
| (d) Sodium Starch Glycolate | 20 | 12 |
| (e) Magnesium Stearate | 5 | 3 |
| | 500 | 300 |

Formulation B

| | mg/tablet | mg/tablet |
|---|---|---|
| (a) Active ingredient | 250 | 250 |
| (b) Lactose | 150 | — |
| (c) Avicel PH 101 ® | 60 | 26 |
| (d) Povidone B.P. | 15 | 9 |
| (e) Sodium Starch Glycolate | 20 | 12 |
| (f) Magnesium Stearate | 5 | 3 |
| | 500 | 300 |

Formulation C

| | mg/tablet |
|---|---|
| Active ingredient | 100 |
| Lactose | 200 |
| Starch | 50 |
| Povidone | 5 |
| Magnesium stearate | 4 |
| | 359 |

The following formulations, D and E, are prepared by direct compression of the admixed ingredients. The lactose used in formulation E is of the direction compression type.

Formulation D

| | mg/capsule |
|---|---|
| Active Ingredient | 250 |
| Pregelatinised Starch NF15 | 150 |
| | 400 |

Formulation E

| | mg/capsule |
|---|---|
| Active Ingredient | 250 |
| Lactose | 150 |
| Avicel ® | 100 |
| | 500 |

Formulation F (Controlled Release Formulation)
The formulation is prepared by wet granulation of the
ingredients (below) with a solution of povidone followed
by the addition of magnesium stearate and compression.

| | | mg/tablet |
|---|---|---|
| (a) | Active Ingredient | 500 |
| (b) | Hydroxypropylmethylcellulose | 112 |
| | (Methocel K4M Premium) ® | |
| (c) | Lactose B.P. | 53 |
| (d) | Povidone B.P.C. | 28 |
| (e) | Magnesium Stearate | 7 |
| | | 700 |

Drug release tales place over a period of about 6-8 hours and was complete after 12 hours.

### Example D

### Capsule Formulations

### Formulation A

A capsule formulation is prepared by admixing the ingredients of Formulation D in Example C above and filing into a two-part hard gelatin capsule. Formulation B (infra) is prepared in a similar manner.

Formulation B

| | mg/capsule |
|---|---|
| (a) Active ingredient | 250 |
| (b) Lactose B.P. | 143 |
| (c) Sodium Starch Glycolate | 25 |
| (d) Magnesium Stearate | 2 |
| | 420 |

Formulation C

| | mg/capsule |
|---|---|
| (a) Active ingredient | 250 |
| (b) Macrogol 4000 BP | 350 |
| | 600 |

Capsules are prepared by melting the Macrogol 4000 BP, dispersing the active. ingredient in the melt and filling the melt into a two-part hard gelatin capsule.

Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

### Formulation E (Controlled Release Capsule)

The following controlled release capsule formulation is prepared by extruding ingredients a, b, and c using an extruder followed by spheronisation of the extrudate and drying. The dried pellets are then coated with release-controlling membrane (d) and filled into a two-piece; hard gelatin capsule.

| | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

### Example E

### Injectable Formulation

| Active ingredient | |
|---|---|
| Sterile, pyrogen free phosphate | 0.200 g |
| buffer (pH 7.0) to 10 ml | |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### Example F

### Intramuscular Injection

| | |
|---|---|
| Active ingredient | 0.20 g |
| Benzyl Alcohol | 0.10 g |
| Glucofurol 75 ® | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore fitter and sealed in sterile 3 ml glass vials (type 1).

### Example G

### Syrup Suspension

| | |
|---|---|
| Active ingredient | 0.2500 g |
| Sorbitol Solution | 1.5000 g |
| Glycerol | 2.0000 g |
| Dispersible Cellulose | 0.0750 g |
| Sodium Benzoate | 0.0050 g |
| Flavour, Peach 17.42.3169 | 0.0125 ml |
| Purified Water q.s. to | 5.0000 ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further Sickening is achieved as required by extra shearing of the suspension.

### Example H

### Suppository

| | mg/suppository |
|---|---|
| Active ingredient (63 μm)* | 250 |
| Hard Fat, BP (Witepsol H15 - Dynamit Nobel) | 1770 |
| | 2020 |

*The active ingredient is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base wait mixing, using a silverson fitted with a cutting head, until a smooth dispersion is achieved. Maintaining the mixture at 45° C. the remaining Witepsol H15 is added to the suspension and stirred to ensure a homogenous mix. The entire suspension is passed through a 250 μm stainless steel screen and, with continuous stirring, is allocated to cool to 40° C. At a temperature of 38° C. to 40° C. 2.02 g of the mixture is filled into suitable plastic moulds. The suppositories are allowed to cool to room temperature.

### Example I

### Pessaries

| | mg/pessary |
|---|---|
| Active ingredient | 250 |
| Anhydrate Dextrose | 380 |
| Potato Starch | 363 |
| Magnesium Stearaie | 7 |
| | 1000 |

The above ingredients are mixed directly and pessaries prepared by direct compression of the resulting mixture.

### REFERENCES

Asano et al., 1986, Mol Pharmacol 29, 244-9.

Ausubel et al. 1995, Current Protocols in Molecular Biology (Wiley Interscience Publishers).

Beggs, 1978, Nature, 275:104-109.

Beindl et al., 1996, Mol Pharmacol, 50, 415-23.

Bellingham et al., 2002, Brain Res Mol Brain Res, 107, 128-36.

Bennett et al., 2000, Curr. Opin. Mol. Ther. 2:420-425.

Berent et al., 1985, Biotech, 3:208.

Berson et al., 2001, Inv. Ophthalmology and Visual Science, 42:S113.

Berson et al., 2002, Science, 295, 1070-3.

Bleasdale et al., 1990, J Pharmacol Exp Ther, 255, 756-68.

Botstein et al., 2001, Science, 229:193-1210.

Brainard et al., 2001, Journal of Neuroscience, 21:6405-6412.

Chow et al., 2001, Trans. Neural. Syst. Rehabil. Eng., 9:86-95.

Cohen et al., 1972, Proc. Natl. Acad. Sci. USA 69, 2110.

Czeisler et al., 1995, The New England Journal of Medicine, 332:6-11.

Derossi et al., 1998, Trends Cell Biol, 8:84-87.

Dixon et al., 1987, Photochem Photobiol, 46, 115-9.

Filipek et al., 2003, Annu. Rev. Physiol, 65:851-79.

Fine et al., 2000, N Engl J Med, 17;342:483-92.

Freedman et al., 1997, Science, 284:502-504.

Gilman et al., 1984, Cell, 36, 577-9.

Gooley et al., 2001, Nat Neurosci 4, 1165.

Hankins et al., 2002, Curr Biol, 12, 191-8.

Hannibal et al., 1997, J. Neurosci, 17:2637-2644.

Hannibal et al., 2002, J. Neurosci, 22:RC191.

Hardie et al., 1991, proceedings of the Royal Society, 245:203-210.

Hastings et al, 1998, International Patent No. WO 98/16643.

Hattar et al., 2003, Nature, 424, 75-81.

Hattar et al., 2002, Science 95, 1065-70.

Hao et al., 1999, J Biol Chem 274, 6,085-6090.

Hillman et al., 1993, Physiological Reviews, 63:668-772.

Huang et al., 2004, Cancer Research, 64, 4294-301.

Hubbard et al., 1958, J Gen Physiol, 41, 501-528.

Inglehearn et al., 1998, Eye, 12:571-579.

Klerman et al., 2002 J. Biol Rhythms, 17, 548-55.

Koyanagi et al., 2004, Proc. Natl. Acad. Sci, 101:6687-91.

Koyanagi et al., 2002, FEBS Lett 531, 525-8.

Liu, et al., 1986, Biochem, 25, 7026-7030.

Lockley et al., 1997, J Clin. Endo. and Metabolism, 82:3763-3770.

Lucas et al., 1999, Science, 284:505-507.

Lucas et al., 2001, Nature Neuroscience, 4:621-626.

Lund et al., 2001, Proc. Natl. Acad. Sci. USA, 98:9942-9947.

Lucas et al., 2003, Science, 299 245-7.

Matsuda et al., 2004, Proc natl. Acad. Sci. USA, 101:16-22.

Mrosovsky et al., 2001, J Biol Rhythms, 16; 585-587.

Newman et al., 2003, Biochem, 42, 12734-8.

Nobile et al., 2000, Gen Physiol Biophys, 19, 207-21.

Panda et al., 2003, Science, 301. 525-527.

Provencio et al., 1998, Journal of Comparative Neurology, 395:417-439.

Provencio et al., 1998, Proc Natl Acad Sci USA, 95, 340-5.

Provencio et al., 2000, J. Neurosci, 20:600-605.

Sambrook et al., 2001, MOLECULAR CLONING: A LABORATORY MANUAL, 3rd edition.

Schlosshauer et al., 1999, Biomedical Microdevices, 2:61-72.

Sekaran et al., 2003, Curr Biol.

Shea et al., 1985, Brain Res, 353, 307-14.

Sherman et al., 1986, Methods In Yeast Genetics, A Laboratory Manual, Cold Spring Harbor, N.Y.

Southern, 1975, J. Mol. Biol. 98:503.

Spencer et al., 1997, Biochem Pharmacol, 54, 809-18.

Stryer, 1991, J. Biol. Chem., 266:10711-10714.

Thapan et al., 2001, J. Physiol, 535, 261-267.

Warren et al., 2003, Eur J Neurosci, 17, 1727-35.

Yokoyama, 2000, Prog Retin Eye Res., 19:385-419.

Zemelman et al., 2002, Neuron, 33, 15-22.

Zrenner et al., 1997, Opthalmic Research, 29:269-280.

Zrenner et al., 1999, Vision Research, 39:2555-2567.

1. (canceled)

2. The method according to claim 39 wherein the photoactivator is a photoactivator which is present in an intrinsically photosensitive retinal ganglion cell (ipRGC).

3. The method according to claim 39 wherein the photoactivator is an opsin.

4. The method according to claim 39 wherein the photoactivator is an opsin and one or more co-factor of the opsin.

5. The method according to claim 39 wherein the photoactivator is a mammalian opsin.

6. The method according to claim 39 wherein the photoactivator is a human opsin.

7. The method according to claim 39 wherein the photoactivator is melanopsin.

8. The method according to claim 39 wherein the photoactivator is produced by expressing a nucleic acid encoding the photoactivator.

9. The method according to claim 8 wherein the nucleic acid comprises:
(i) a nucleotide sequence of SEQ ID NO: 1; or
(ii) a nucleotide sequence which encodes an amino acid sequence with more than 50% identity to the deduced amino acid sequence of SEQ ID NO:1 or a nucleotide sequence which hybridises to the nucleotide sequence of SEQ ID NO:1 under stringent, or moderately stringent conditions; or
(iii) a nucleotide sequence which encodes an amino acid sequence with more than 20% identity to a sequence of at least 200 amino acids of bovine rod opsin; or
(iv) a fragment of the nucleotide sequence of SEQ ID NO:1 encoding a polypeptide fragment effective to induce photosensitivity in a retinal ganglion cell (RGC).

10. The method according to claim 8 wherein the nucleic acid comprises the nucleic acid encoding a photoactivator in a vector.

11. The method according to claim 10 wherein the vector is an expression vector.

12. The method according to claim 10 wherein the vector is suitable for replication in a mammalian cell.

13. The method according to claim 10 wherein the vector is a viral vector.

14. The method according to claim 10 wherein the viral vector is an adeno-associated viral vector.

15. The method according to claim 8 wherein the nucleic acid encodes a photoactivator and one or more proteins involved in the photo-transduction cascade.

16. A vector comprising a nucleic acid encoding a photoactivator.

17. The vector according to claim 16 wherein the photoactivator is an opsin.

18. The vector according to claim 16 wherein the photoactivator is an opsin and one or more co-factor of the opsin.

19. The vector according to claim 16 wherein the photoactivator is a mammalian opsin.

20. The vector according to claim 16 wherein the photoactivator is a human opsin.

21. The vector according to claim 16 wherein the photoactivator is melanopsin.

22. The vector according to claim 16 wherein the nucleic acid has a nucleotide sequence of SEQ ID NO: 1.

23. The vector according to claim 16 wherein the vector is an expression vector.

24. The vector according to claim 16 wherein the vector is suitable for replication in a mammalian cell.

25. The vector according to claim 16 wherein the vector is a viral vector.

26. The vector according to claim 16 wherein the viral vector is an adeno-associated viral vector.

27. A neuronal cell comprising a nucleic acid as defined in claim 9, wherein the neuronal cell is capable of expressing a photoactivator.

28. A neuronal cell comprising a photoactivator as defined in claim 2.

29. The neuronal cell according to claim 27 wherein the neuronal cell is a retinal ganglion cell (RGC).

30. (canceled)

31. (canceled)

32. (canceled)

33. (canceled)

34. The composition according to claim 38 wherein the medicament is in a form adapted for delivery into vitreal space.

35. The composition according to claim 38 wherein the medicament is in a form which is compatible with the vitreous humor/vitreous body.

36. A pharmaceutical composition comprising a photoactivator and/or a nucleic acid and/or a vector and/or a neuronal cell, and a pharmaceutically acceptable carrier or exipient, the photoactivator and/or the nucleic acid and/or the vector and/or the neuronal cell being present in an amount effective to induce photosensitivity in one or more neuronal cell in an individual.

37. The pharmaceutical composition according to claim 36 wherein the neuronal cell is a retinal ganglion cell (RGC).

38. A pharmaceutical composition comprising a photoactivator and/or a nucleic acid and/or a vector and/or a retinal ganglion cell (RGC), and a pharmaceutically acceptable carrier or exipient, the photoactivator and/or the nucleic acid and/or the vector and/or the retinal ganglion cell (RGC) being present in an amount effective to restore sight and/or alleviate blindness in an individual.

39. A method of inducing photosensitivity in one or more neuronal cell comprising inducing production of a photoactivator that is capable of activating a photo-transduction cascade in a neuronal cell in response to light.

40. The method according to claim 39 wherein production of a photoactivator is induced in one or more neuronal cell by expressing a nucleic acid encoding a photoactivator in one or more neuronal cell.

41. The method according to claim 39 wherein the nucleic acid encoding a photoactivator is introduced into one or more neuronal cell.

42. The method according to claim 39 wherein the nucleic acid encoding a photoactivator is usually present in the genome of a neuronal cell.

43. The method according to claim 39 wherein the neuronal cell is a retinal ganglion cell (RGC).

44. A method of restoring sight and/or alleviating blindness in an individual comprising inducing production of a photoactivator in one or more retinal ganglion cell (RGC) that is capable of activating a photo-transduction cascade in a retinal ganglion cell (RGC) in response to light.

45. A method of inducing photosensitivity in one or more neuronal cell, the method comprising administering an effective amount of a pharmaceutical composition according to claim 36 to a subject in need thereof.

46. A method of inducing photosensitivity in one or more neuronal cell in vitro, the method comprising introducing an effective amount of a photoactivator and/or a nucleic acid and/or a vector into one or more neuronal cell.

47. The method according to claim 46 wherein the neuronal cell is a retinal ganglion cell (RGC).

48. A method of restoring sight and/or alleviating blindness in an individual, the method comprising administering an effective amount of a pharmaceutical composition according to claim 36 to a subject in need thereof.

49. A method of inducing photosensitivity in one or more neuronal cell of an individual, the method comprising the steps of:
(i) introducing a photoactivator and/or a nucleic acid and/or a vector into one or more neuronal cell in vitro so that photosensitivity is induced in the one or more neuronal cell; and
(ii) administering an effective amount of the resulting one or more neuronal cell in which photosensitivity has been induced into a suitable site in the individual.

50. A method of restoring sight and/or alleviating blindness in an individual, the method comprising the steps of:
(i) introducing a photoactivator and/or a nucleic acid and/or a vector into one or more retinal ganglion cell (RGC) in vitro so that photosensitivity is induced in the one or more retinal ganglion cell (RGC); and
(ii) administering an effective amount of the resulting one or more retinal ganglion cell (RGC) in which photosensitivity has been induced into one or both eye(s) of an individual.

51. A method of inducing photosensitivity in one ore more neuronal cell in an individual, the method comprising the step of administering an effective amount of one or more neuronal cell according to claim 27 to an individual.

52. A method of restoring sight and/or alleviating blindness in an individual, the method comprising the step of administering an effective amount of one or more retinal ganglion cell (RGC) according to claim 29 to an individual.

53. A neuronal cell comprising a vector according to claim 16, wherein the neuronal cell is capable of expressing a photoactivator.

54. The neuronal cell according to claim 54 wherein the neuronal cell is a retinal ganglion cell (RGC).
